(19) United States
(12) Patent Application Publication
(10) Pub. No.: US 2020/0079786 A1
(43) Pub. Date: Mar. 12, 2020

(54) ANALOGS OF ADAMANTYLUREAS AS SOLUBLE EPOXIDE HYDROLASE INHIBITORS

(71) Applicant: UNIVERSITAT DE BARCELONA, Barcelona (ES)

(72) Inventors: Santiago Vázquez Cruz, Barcelona (ES); Elena Valverde Murillo, Banyeres del Penedes (ES); Rosana Leiva Martínez, Barcelona (ES); Manuel Vázquez Carrera, Barcelona (ES); Sandra Codony Gisbert, Barcelona (ES)

(21) Appl. No.: 15/746,791

(22) PCT Filed: Jul. 25, 2016
(86) PCT No.: PCT/EP2016/067620

§ 371 Date: Jan. 22, 2018

(30) Foreign Application Priority Data
Jul. 28, 2015 (EP) 15178618.3

(51) Int. Cl. C07D 493/08

(52) U.S. Cl. CPC C07D 493/08

(57) ABSTRACT

N-(2-oxaadamantan-1-yl)ureas of formula I, where R3 is H, C₁-C₃ alkyl, cyclohexyl or phenyl; R is —[CH₂]ₙ—Y; n is 0-15; in —[CH₂]ₙ— 0-n/3 of the methylene groups are optionally replaced by non adjacent oxygen atoms; and Y is a 3- or 4-substituted phenyl, a 3- or 4-substituted cyclohexyl, a N-substituted piperidin-4-yl, a N-substituted piperidin-3-yl, a di- or tri-fluorosubstituted phenyl, 4-chloro-3-trifluoromethylphenyl, 3-chloro-4-trifluoromethylphenyl, 4-fluoro-3-trifluoromethylphenyl, or 3-fluoro-4-trifluoromethylphenyl; have epoxide hydrolase (sEH) inhibitory activities similar to those of their N-(adamantan-1-yl)urea analogs. Thus, compounds I are useful as API for the treatment of sEH mediated diseases. Besides, in general, compounds I have higher water solubilities and lower melting points, what make them more promising from the point of view of pharmacokinetics and formulation.

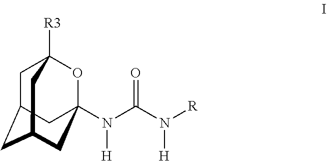

The present invention relates to the field of pharmaceutical products for human and veterinary medicine, particularly to soluble epoxide hydrolase (sEH) inhibitors and their therapeutic indications.

### BACKGROUND ART

A total of more than 100 patent publications have described multiple classes of sEH inhibitors, based on different chemical structures, such as amides, thioamides, ureas, thioureas, carbamates, acyl hydrazones and chalcone oxides (cf. e.g. H. C. Shen, “Soluble epoxide hydrolase inhibitors: a patent review”, Expert. Opin. Ther. Patents 2010, vol. 20, pp. 941-956, a review with 149 references). sEH inhibition has been associated to various beneficial biological effects, that may be translated into therapeutic treatment for hypertension, atherosclerosis, pulmonary diseases, kidney diseases, stroke, pain, neuropathic pain, inflammation, pancreatitis, immunological disorders, eye diseases, cancer, obesity, diabetes, metabolic syndrome, preeclampsia, anorexia nervosa, depression, erectile dysfunction, wound healing, NSAID-induced ulcers, emphysema, scrapie and Parkinson's disease (cf. e.g. H. C. Shen and B. D. Hammock, “Discovery of inhibitors of soluble epoxide hydrolase: A target with multiple potential therapeutic indications”, J. Med. Chem. 2012, vol. 55, pp. 1789-1808, a review with 117 references).

Despite the high inhibitory activity of many of the reported sEH inhibitory compounds, until now no sEH inhibitor has reached the market, what illustrates the difficulty of developing sEH inhibitors as human active pharmaceutical ingredients (API). Some of the development limitations are: lack of selectivity, chemical and metabolical instability, and inappropriate physical properties, especially low water solubility. Therefore, there is a need for development of new sEH inhibitory compounds that, having an acceptable inhibitory activity, overcome some of these limitations.

### SUMMARY OF INVENTION

Inventors have found that by the simultaneous triple selection of: (i) urea as the core chemical functional group; (ii) the adamantan-1-yl group, optionally 3-substituted, as one of the N-substituents of urea; and (iii) the replacement of the 2-methylene biradical of the adamantan-1-yl moiety by an oxygen atom, new sEH inhibitors are obtained that, compared with their adamantyl analogs, have similar activity, improved water solubility, and lower melting points.

Many N-(adamantan-1-yl)ureas of general formula I′ have been reported to be sEH inhibitors. Virtually all of them are unsubstituted in position 3 of the adamant-1-yl moiety, i.e. they have R3=H in their formula I′.

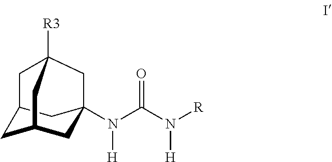

A vast majority of the specifically reported 3-unsubstituted N-(adamantan-1-yl)ureas with sEH inhibitor activity are disclosed in the following five patent documents, here referred to as Pat-Doc1 to Pat-Doc 5:

Pat-Doc 1: US 20050164951 A1; “Inhibitors for the soluble epoxide hydrolase”; University of California; 117 pp.; Chemical Abstracts Service Accession Number (CAS AN)=2005:672863. This documents specifically discloses about 130 sEH inhibitors encompassed by formula I′.

Pat-Doc 2: WO 2006045119 A2; “Improved inhibitors for the soluble epoxide hydrolase”; University of California; 179 pp.; CAS AN=2006:386356. This document specifically discloses about 110 sEH inhibitors encompassed by formula I′ which are not disclosed in Pat-Doc 1.

Pat-Doc 3: WO 2007106525 A1; “Piperidinyl, indolyl, pirinidyl, morpholinyl and benzimidazolyl urea derivatives as inhibitors of soluble epoxide hydrolase for the treatment of hypertension, inflammations and other diseases”; University of California & Arete Therapeutics; 116 pp.; CAS AN=2007:1061416. This document specifically discloses 48 sEH inhibitors encompassed by formula I′ which are not disclosed neither in Pat-Doc 1 nor in Pat-Doc 2.

Pat-Doc 4: WO 2008040000 A2; “Soluble epoxide hydrolase inhibitors”; Arete Therapeutics; 73 pp.; CAS AN=2008:411908. This document specifically discloses 12 sEH inhibitors encompassed by formula I′ which are not disclosed in any of the other Pat-Doc documents.

Pat-Doc 5: WO 2008051875 A2; “Soluble epoxide hydrolase inhibitors”; Arete Therapeutics; 58 pp.; CAS AN=2008:529196. This document specifically discloses 6 sEH inhibitors encompassed by formula I′ which are not disclosed in any of the other Pat-Doc documents.

Although hundreds of N-(adamantan-1-yl)ureas of the general formula I′ with R3=H have been specifically disclosed as sEH inhibitors, many of them in the aforementioned five Pat-Doc documents, only few are in pharmaceutical development. Among the latter the three below have been considered especially relevant by inventors, and inventors have synthesized and tested the analog N-(2-oxaadamantan-1-yl)ureas of these three N-(adamantan-1-yl)ureas for illustrative comparative purposes.

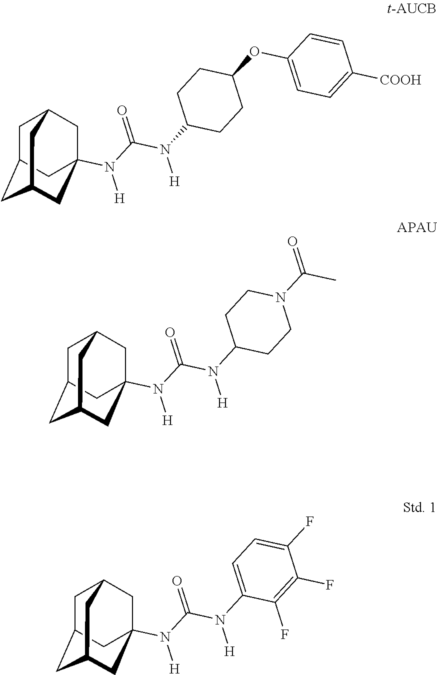

An aspect of the present invention relates to the provision of compounds of formula I

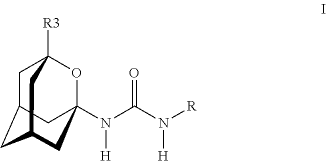

or stereoisomers or pharmaceutically acceptable salts thereof, wherein:
R3 is a radical selected from the group consisting of H, C₁-C₃ alkyl, cyclohexyl and phenyl;
R is a radical —[CH₂]ₙ—Y, wherein n is an integer between 0 and 15, and in the —[CH₂]ₙ-biradical an integer between 0 and n/3 of the methylene groups are optionally replaced by oxygen atoms in such a way that there are not two oxygen atoms which are adjacent;
Y is a radical selected from the group consisting of: phenyl; a substituted phenyl; cyclohexyl; a substituted cyclohexyl; a piperidinyl; a substituted piperidinyl; a C- or N-radical from a 5- or 6-membered aromatic heterocycle; and a C- or N-radical from a 5- or 6-membered aromatic heterocycle which is fused with a benzene ring;

with the proviso that I is not 1-(2-oxaadamantan-1-yl)-3-(3,4-dichlorophenyl)urea.

The compound 1-(2-oxaadamantan-1-yl)-3-(3,4-dichlorophenyl)urea is not considered part of the present invention because its preparation was mentioned in U.S. Pat. No. 3,539,626 (published in 1970, with priority of 1965), where some substituted ureas and thioureas are disclosed, saying that they have antibacterial activity (although no experimental data are provided). It is noteworthy that, of the more than twenty specific compounds which are prepared in this document, this is the only one having the 2-oxaadamantan-1-yl moeity, all the others having the adamantan-1-yl moeity.

In particular embodiments, Y is a radical selected from the group consisting of:
di- and tri-substituted phenyl radicals, wherein the two or three substituents, equal or different, are independently selected from the group consisting of F, Cl, SF₅, CF₃, OH, OCF₃, C₁-C₃ alkyl, and (C₁-C₃)—OCO;
a C- or N-radical from a 5- or 6-membered aromatic heterocycle, having in the cycle one, two or three atoms of N, S or O;
a C- or N-radical from a 5- or 6-membered aromatic heterocycle having in the cycle one, two or three atoms of N, S or O, which is fused with a benzene ring; and
radicals having one of the following four general formulas, wherein bonds crossing positions 3 and 4 of the phenyl and cyclohexyl rings mean substitution either in position 3 or in position 4 of the radical ring;

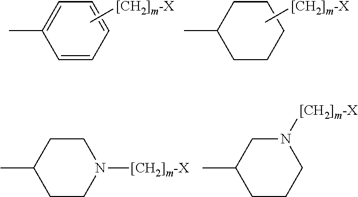

wherein m is an integer between 0 and 15, and in the —[CH₂]ₘ— biradical an integer between 0 and m/3 methylene groups are optionally replaced by oxygen atoms in such a way that there are not two oxygen atoms which are adjacent;
X being a radical selected from the group consisting of:
H, F, Cl, SF₅, CF₃, OCF₃, OH, CN, COOH, C₁-C₃ alkyl, (C₁-C₃ alkyl)CO, (C₁-C₃ alkyl)SO₂;
phenyl, phenoxy, benzoyl, mono-substituted phenyl, mono-substituted benzoyl and mono-substituted phenoxy wherein the substituent is selected from the group consisting of F, Cl, CHO, COCH₃, COOH, and H₂NSO₂;
(C₁-C₁₅ linear alkyl)O, (C₄-C₁₅ linear alkyl)CO, (C₁-C₁₅ linear alkyl)OCO, (C₁-C₁₅ linear alkyl)NHCO, (C₁-C₁₅ linear alkyl)CONH, (C₄-C₁₅ linear alkyl)SO₂, (C₁-C₁₅ linear alkyl)NHSO₂, (C₁-C₁₅ linear alkyl)SO₂NH;
(C₃-C₆ carbocyclyl)O, (C₃-C₆ carbocyclyl)CO, (C₃-C₆ carbocyclyl)OCO, (C₃-C₆ carbocyclyl)NHCO, (C₃-C₆ carbocyclyl)CONH, (C₃-C₆ carbocyclyl)SO₂, (C₃-C₆ carbocyclyl)NHSO₂ (C₃-C₆ carbocyclyl)SO₂NH;
(5/6-membered-N/O-heterocyclyl)O, (5/6-membered-N/O-heterocyclyl)CO, (5/6-membered-N/O-heterocyclyl)OCO, (5/6-membered-N/O-heterocyclyl)NHCO, (5/6-membered-N/O-heterocyclyl)CONH, (5/6-membered-N/O-heterocyclyl)SO₂, (5/6-membered-N/O-heterocyclyl)NHSO₂, and (5/6-membered-N/O-heterocyclyl)SO₂NH; wherein 5/6-membered-N/O-heterocyclyl is a C- or N-radical from a 5- or 6-membered heterocycle, the heterocycle being aromatic or non-aromatic, the heterocycle having in the cycle one, two or three atoms of N, S or O; and wherein the 5/6-membered-N/O-heterocyclyl radical is optionally substituted by one or two substituents, equal or different, independently selected from the group consisting of F, Cl, CF₃, C₁-C₃ alkyl, and (C₁-C₃ alkyl)NH.

In particular embodiments, Y is a radical selected from the group consisting of:

di- and a tri-fluorosubstituted phenyl radicals;

4-chloro-3-trifluoromethylphenyl;

3-chloro-4-trifluoromethylphenyl;

4-fluoro-3-trifluoromethylphenyl,

3-fluoro-4-trifluoromethylphenyl; and

radicals having the above-mentioned four formulas, with an X that is a radical selected from the group consisting of: H, F, Cl, CF₃, OCF₃, OH, CN, COOH, (C₁-C₁₅ linear alkyl)O, (C₁-C₁₅ linear alkyl)CO, (C₁-C₁₅ linear alkyl)OCO, phenyl, phenoxy, mono-substituted phenyl and mono-substituted phenoxy, wherein the substituent is COOH, Cl or H₂NSO₂; (C₁-C₁₅ linear alkyl)NHCO, (C₁-C₁₅ linear alkyl)CONH, (C₁-C₁₅ linear alkyl)SO₂, (C₁-C₁₅ linear alkyl)NHSO₂, (C₁-C₁₅ linear alkyl)SO₂NH; (5/6-membered-N/O-heterocyclyl)O, (5/6-membered-N/O-heterocyclyl)CO, (5/6-membered-N/O-heterocyclyl)OCO, (5/6-membered-N/O-heterocyclyl)-NHCO, (5/6-membered-N/O-heterocyclyl)CONH; (5/6-membered-N/O-heterocyclyl)SO₂, (5/6-membered-N/O-heterocyclyl)NHSO₂, and (5/6-membered-N/O-heterocyclyl)SO₂NH; wherein 5/6-membered-N/O-heterocyclyl now means a C-radical or a N-radical from any 5- or 6-membered heterocycle, the heterocycle being aromatic or non-aromatic, and the heterocycle having in the cycle either one N atom, or two N atoms, or simultaneously one N atom and one O atom.

In particular embodiments, compounds I have an integer n between 0 and 3, and consequently only one methylene group is optionally replaced by an oxygen atom. In another particular embodiment n is 0, and consequently R═Y.

In particular embodiments compounds I have an Y of the following formula.

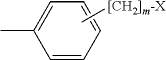

In other particular embodiments compounds I have an Y of the following formula.

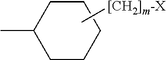

In other particular embodiments compounds I have an Y of the following formula.

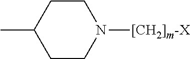

More particular embodiments are those where Y have the three aforementioned general formula where integer m is between 0 and 3; and most particular those where m=0.

In particular embodiments of the aforementioned compounds, X is a radical selected from the group consisting of: H, F, Cl, CF₃, OCF₃, OH, CN, COOH, (C₁-C₅ linear alkyl)O, (C₁-C₅ linear alkyl)CO, (C₁-C₅ linear alkyl)OCO, (C₁-C₅ linear alkyl)NHCO, (C₁-C₅ linear alkyl)CONH, (C₁-C₅ linear alkyl)SO₂, (C₁-C₅ linear alkyl)NHSO₂, (C₁-C₅ linear alkyl)SO₂NH, 2-pyridinyl, 3-pyridynyl, 4-pyridynyl, 4-morpholinyl, phenyl, phenoxy, a mono-substituted phenyl and a mono-substituted phenoxy, whose substitution in the two latter cases is done by a radical selected from the group consisting of COOH, Cl and H₂NSO₂. Even more particular are the following specific compounds:
1-(2-oxaadamantan-1-yl)-3-(1-acetylpiperidin-4-yl)urea; and
trans-1-(2-oxaadamantan-1-yl)-3-[4-(4-carboxyphenoxy)cyclohexyl]urea.

Particular embodiments are also those compounds of formula I where Y is a tri-fluorosubstituted phenyl radical, 4-chloro-3-trifluoromethylphenyl, 3-chloro-4-trifluoromethylphenyl, 4-fluoro-3-trifluoromethylphenyl, or 3-fluoro-4-trifluoromethylphenyl. Even more particular are the following specific compounds:
1-(2-oxaadamantan-1-yl)-3-(2,3,4-trifluorophenyl)urea;
1-(3-methyl-2-oxaadamantan-1-yl)-3-(2,3,4-trifluorophenyl)urea;
1-(3-ethyl-2-oxaadamantan-1-yl)-3-(2,3,4-trifluorophenyl)urea;
1-(3-cyclohexyl-2-oxaadamantan-1-yl)-3-(2,3,4-trifluorophenyl)urea;
1-(3-phenyl-2-oxaadamantan-1-yl)-3-(2,3,4-trifluorophenyl)urea;

Other aspect of the present invention relates to pharmaceutical compositions comprising therapeutically effective amounts of compounds of formula I, or stereoisomers or pharmaceutically acceptable salts thereof, and adequate amounts of pharmaceutically acceptable excipients. Pharmacy in the context of the present invention relates both to human medicine and veterinary medicine.

From the results of the accompanying illustrative examples and by analogy with compounds of formula I′ of prior art, inventors have concluded that compounds of formula I are sEH inhibitors. Thus, other aspect of the present invention relates to compounds of formula I, or stereoisomer or pharmaceutically acceptable salts thereof, for use in the treatment of sEH mediated diseases. In particular embodiments the sEH mediated diseases are hypertension, atherosclerosis, pulmonary diseases, kidney diseases, stroke, pain, neuropathic pain, inflammation, pancreatitis, immunological disorders, eye diseases, cancer, obesity, diabetes, metabolic syndrome, preeclampsia, anorexia nervosa, depression, erectile dysfunction, wound healing, NSAID-induced ulcers, emphysema, scrapie and Parkinson's disease. In other words, the present invention is related to methods of treatment of human patients suffering from a sEH mediated disease, by administration of pharmaceutical compositions comprising compounds of formula I and adequate amounts of pharmaceutically acceptable excipients. Methods for treatment of the aforementioned particular sEH mediated diseases are particular embodiments of the present invention. And the aforementioned pharmaceutical compositions also form part of the present invention.

As compounds of formula I have never been disclosed for use in animal therapy, including human therapy, an aspect of the present invention relates to compounds of formula I, or stereoisomers or pharmaceutically acceptable salts thereof, for use as active pharmaceutical ingredients.

According to other aspect of the present invention, there are provided two alternative processes for the preparation of compounds of formula I from amines of formula II, as shown in the accompanying scheme.

According to the first alternative, amine of formula II, preferably in the form of a salt such as the hydrochloride, is reacted with isocyanate of formula OCN—R, in an inert solvent such as dichloromethane (DCM), and in the presence of a base such as triethylamine. According to the second alternative, in a first step (a) amine of formula II, preferably in the form of a salt, is converted into isocyanate of formula IV by reaction with an (NH₂→NCO) converting reagent such as triphosgene, and in an inert solvent such as DCM. In a second step (b), amine of formula R—NH₂ is reacted with isocyanate of formula IV, a chemical transformation analogous to the one of the first alternative.

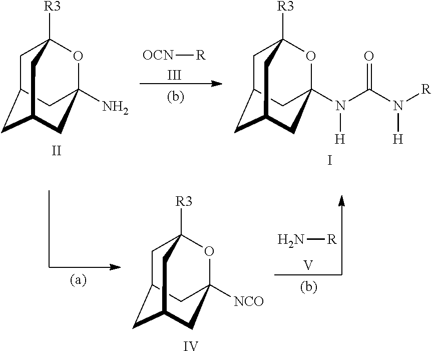

As a third alternative, not shown in the scheme, some compounds I with a given substituent R may be obtained from compounds I with a substituent R′, R′ being a precursor or a R-protected group. In the examples this is illustrated by the preparation of a compound I with R=piperidin-4-yl by palladium-catalyzed hydrogenation of a compound I with R′=benzylpiperidin-4-yl.

Amines of formula II are either commercially available or obtainable from known starting materials as disclosed in the art (cf. e.g. M. D. Duque et al., “Synthesis and pharmacological evaluation of (2-oxaadamantan-1-yl)amines”; Bioorg. Med. Chem. 2009, vol. 17, pp. 3198-3206). Isocyanates of formula OCN—R and amines of formula R—NH₂ are either commercially available or obtainable as disclosed in the art, e.g. in the aformentioned documents Pat-Doc 1 and Pat-Doc 2.

IC₅₀ values of Table 1 illustrate that the N-(2-oxaadamantan-1-yl)ureas of the present invention have an sEH inhibitory activity similar to their analog N-(adamantan-1-yl)ureas which are disclosed in the art as sEH inhibitors. In fact, compounds Iₐ to I_{g} have IC₅₀ values lower than 22 nM, which represents an acceptable activity for the target. Therefore, the introduction of an R3 radical in the 3 position of the 2-oxaadamantyl moiety (illustrated by compounds I_{b} to Iₑ) does not involve a decrease in activity. It is noteworthy than compound Iₐ has an IC₅₀ value of 2.58 nM, which is significantly lower than the one of its parent adamantyl analog (7.74 nM, Std 1).

Experimental value of solubility (S) for compound Iₐ in Table 1 is higher than S for compound Std 1. In general, the N-(2-oxaadamantan-1-yl)ureas of the present invention have water solubilities similar or higher than their analog N-(adamantan-1-yl)ureas which are disclosed in the art as sEH inhibitors, what is in accordance with their calculated values of log P, shown in same table as c log P. Results in Table 1 illustrate that compounds Iₐ to Iₑ have melting points substantially lower than their analog N-(adamantan-1-yl)ureas which are disclosed in the art as sEH inhibitors. Since it is known (cf. e.g. S. H. Hwang et al., “Orally bioavailable potent sEH inhibitors”; J. Med. Chem. 2007, vol. 50, pp. 3825-3840) that N-(adamantan-1-yl)ureas that are both poorly soluble in water and have a stable crystal structure as indicated by a high melting point are difficult to formulate, the physicochemical properties of the N-(2-oxaadamantan-1-yl)ureas of the present invention are good both from the point of view of pharmacokinetics and formulation. This fact, together with their acceptable sEH inhibitory activities, makes the N-(2-oxaadamantan-1-yl)ureas of the present invention promising API for the treatment of sEH mediated diseases.

The in vitro results of Example 24 and Table 2 show that compounds Iₐ and I_{g} behave in a manner similar to the compound used as comparative standard, in the reduction of endoplasmic reticulum (ER) stress induced by palmitate. Since it has been suggested that ER stress is involved in the appearance of insulin resistance, inflammation, neuropathic pain, metabolic syndrome and related disorders, the facts that sEH inhibitors of formula I significantly reduce ER stress, that they are not cytotoxic, and that they can pass the cell membrane, also contribute to the conclusion that the N-(2-oxaadamantan-1-yl)ureas of the present invention are promising API for the treatment of sEH mediated diseases.

According to Example 25, and the corresponding results in Table 3, inventors have found that selected compounds of the present invention present appropriate sEH inhibition activity values in pancreatic rat cells (AR42j), what makes them promising API for treatment of e.g. pancreatitis.

According to Example 26, and the corresponding results in Table 3, inventors have found that selected compounds of the present invention present relative low cytotoxicity values in human liver cells, what makes them promising for human treatment.

According to Example 27, and the corresponding results in Table 3, inventors have found that selected compounds of the present invention are likely able to cross the blood-brain barrier, what makes them promising for treating CNS diseases or disorders.

The epoxidation of arachidonic acid (AA) by selected cytochrome P450 epoxygenases generates epoxyeicosatrienoic acids (EETs). These EETs show anti-inflammatory, antihypertensive, analgesic, angiogenic, and antiatherosclerotic effects in rodents and humans. sEH converts EETs to their corresponding dihydroxyeicosatrienoic acids (DHETs), whereby the biological effects of EETs are diminished, eliminated, or altered. Among the P450 enzymes, it is known that CYP2C19 and CYP1A2 have the highest formation rate of EETs from AA (cf. A. A. El-Sherbeni et al. “Repurposing resveratrol and fluconazole to modulate human cytrochrome P450-mediated arachidonic acid metabolites”, Molecular Pharmaceutics 2016, vol. 13, pp. 1278-1288). For this reason, a highly desirable aspect of any new sEH inhibitor is selectivity in front of CYP2C19 and CYP1A2. Some selected compounds of the present invention (Ia, Ig, If, Io, Is, Iu, Iv, and Ix) were tested for their inhibition at 1 μM of the human cytochrome P450 enzymes CYP1A2 and CYP2C19, and all displayed very weak inhibition (≤6%).

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

Analytical Methods
Melting points were determined in open capillary tubes with a MFB 595010 M Gallenkamp melting point apparatus.
Infrared (IR) spectra, using the attenuated total reflectance (ATR) technique, were run on a Perkin-Elmer Spectrum RX I spectrophotometer. Absorption values are expressed as wavenumbers (cm¹); only significant absorption bands are given.
Gas Chromatography/Mass Spectrometry (GC/MS) analysis was carried out in an inert Agilent Technologies 5975 gas chromatograph equipped with an Agilent 122-5532 DB-5MS 1b (30 m×0.25 mm) capillary column with a stationary phase of phenylmethylsilicon (5% diphenyl-95% dimethylpolysiloxane), using the following conditions: initial temperature of 50° C. (1 min), with a gradient of 10° C./min up to 300° C., and a temperature in the source of 250° C., Solvent Delay (SD) of 4 min and a pressure of 7.35 psi. The direct insertion proble (DIP) technique was used. The electron impact (70 eV) or chemical ionization (CH4) techniques were used. Only significant ions are given: those with higher relative ratio, except for the ions with higher m/e values.

Elemental analyses was carried out at the Mycroanalysis Service of the IIQAB (CSIC, Barcelona, Spain) with a Carlo Erba model 1106 analyzer.
Column chromatography was performed on silica gel 60 Å C. C (35-70 mesh, SDS, ref 2000027). Thin-layer chromatography was performed with aluminum-backed sheets with silica gel 60 F254 (Sigma-Aldrich, ref 60805), and spots were visualized with UV light, 1% aqueous solution of KMnO₄ and/or iodine.
Analytical grade solvents were used for crystallization, while pure for synthesis solvents were used in the reactions, extractions and column chromatography.
The analytical samples of all of the new compounds which were subjected to pharmacological evaluation possess a purity ≥95% as evidenced by their elemental analyses.

### Example 1a: Preparation of 1-(2-oxaadamantan-1-yl)-3-(2,3,4-trifluorophenyl)urea, Iₐ

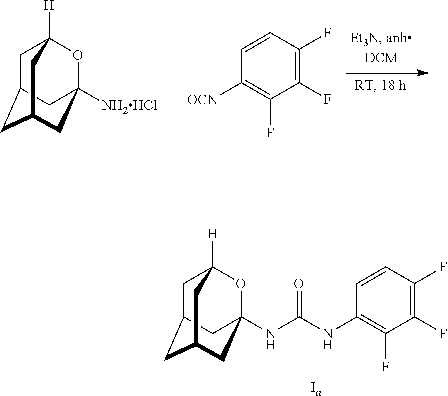

In a round-bottom flask equipped with a stir bar under nitrogen atmosphere 1.2 eq. of (2-oxaadamantan-1-yl)amine hydrochloride was added to anh. dichloromethane (DCM) (˜110 mM). To this suspension 1.0 eq. of 2,3,4-trifluorophenyl isocyanate followed by 7 eq. of triethylamine (TEA) was added. The reaction mixture was stirred at room temperature overnight. Then the solvent was removed under vacuo and the resulting crude was purified by column chromatography (SiO₂, Hexane/Ethylacetate mixture) of the crude and evaporation in vacuo of the appropriate fractions gave the urea Iₐ (163 mg, 94% yield) as a white solid, mp 196-198° C. IR (ATR): 3300-2800 (3293, 3232, 3127, 2933, 2857), 1702, 1640, 1621, 1563, 1509, 1489, 1471, 1446, 1373, 1349, 1340, 1317, 1294, 1257, 1239, 1227, 1200, 1165, 1117, 1099, 1080, 1020, 996, 976, 963, 932, 912, 884, 840, 805, 788, 757, 683, 653 cm¹. MS (DIP), m/e (%): 179 (11), 172 (18), 149 (97), 148 (100), 146 (36), 121 (12), 120 (10), 118 (13), 111 (11), 95 (17), 94 (26), 93 (11), 79 (20), 68 (18). Anal. Calcd for C₁₆H₁₇F₃N₂O₂.0.05 Pentane: C 59.15, H 5.37, F 17.28, N 8.49. Found: C 59.00, H 5.60, F 17.22, N 8.57.

### Example 1b: Preparation of 1-(3-methyl-2-oxaadamantan-1-yl)-3-(2,3,4-trifluorophenyl)urea, I_{b}

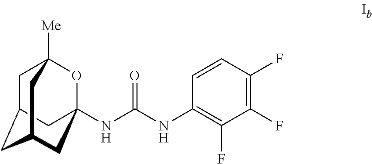

Using (3-methyl-2-oxaadamant-1-yl)amine in a process analogous to the one of Example 1a, the title compound was obtained in a 93% yield. Mp 195-197° C. IR (ATR): 3300-2800 (3270, 3227, 3128, 2976, 2927, 2856), 1701, 1641, 1622, 1564, 1509, 1492, 1471, 1373, 1341, 1322, 1301, 1286, 1256, 1228, 1213, 1200, 1171, 1136, 1106, 1090, 1072, 1034, 1006, 991, 972, 959, 921, 899, 885, 804, 788, 755, 682, 670, 652 cm⁻¹. MS (DIP), m/e (%): 172 (13), 150 (14), 149 (100), 148 (80), 147 (25), 109 (10), 108 (14), 107 (11), 95 (10), 93 (25). Anal. Calcd for C₁₇H₁₉F₃N₂O₂.0.05H₂O: C 59.84, H 5.64, F 16.70, N 8.21. Found: C 59.91, H 5.90, F 16.52, N 8.22.

### Example Ic: Preparation of 1-(3-ethyl-2-oxaadamantan-1-yl)-3-(2,3,4-trifluorophenyl)urea, I_{c}

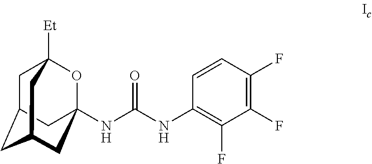

Using (3-ethyl-2-oxaadamantan-1-yl)amine in a process analogous to the one of Example 1a, the title compound was obtained in a 96% yield. Mp 165-166° C. IR (ATR): 3300-2800 (3288, 3238, 3128, 2970, 2927, 2850), 1702, 1641, 1622, 1563, 1509, 1471, 1371, 1341, 1322, 1301, 1254, 1227, 1209, 1172, 1091, 1010, 996, 965, 939, 921, 896, 803, 788, 755, 669, 653 cm⁻¹. MS (DIP), m/e (%): 354 (M⁺, 5), 148 (14), 146 (100), 94 (10), 93 (10). Anal. Calcd for C₁₈H₂₁F₃N₂O₂.0.01EtOAc: C 60.99, H 5.98, F 16.04, N 7.89. Found: C 60.97, H 6.06, F 16.23, N 7.84.

### Example Id: Preparation of 1-(3-cyclohexyl-2-oxaadamantan-1-yl)-3-(2,3,4-trifluorophenyl)urea, I_{d}

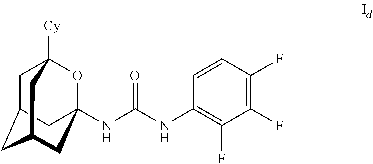

Using (3-cyclohexyl-2-oxaadamantan-1-yl)amine in a process analogous to the one of Example 1a, the title compound was obtained in a 94% yield. Mp 193-195° C. IR (ATR): 3300-2800 (3309, 3227, 3107, 2925, 2855), 1681, 1622, 1537, 1513, 1470, 1326, 1300, 1256, 1234, 1211, 1084, 1061, 1014, 994, 975, 892, 853, 825, 809, 763, 702, 678, 655 cm⁻¹. MS (DIP), m/e (%): 408 (M⁺, 5), 178 (37), 176 (21), 172 (19), 152 (23), 148 (11), 147 (100), 135 (16), 120 (10), 110 (12), 95 (13), 94 (19), 93 (12), 83 (15), 81 (11), 67 (11), 55 (16). Anal. Calcd for C₂₂H₂₇F₃N₂O₂.0.60MeOH: C 63.47, H 6.88, N 6.55. Found: C 63.44, H 7.17, N 6.63.

### Example Ie: Preparation of 1-(3-phenyl-2-oxaadamantan-1-yl)-3-(2,3,4-trifluorophenyl)urea, Iₑ

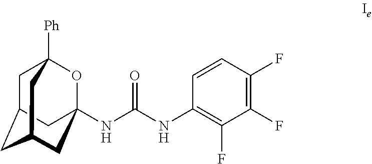

Using (3-phenyl-2-oxaadamantan-1-yl)amine in a process analogous to the one of Example 1a, the title compound was obtained in a 70% yield. Mp 150-152° C. IR (ATR): 3300-2800 (3312, 3238, 3118, 2922, 2856), 1697, 1621, 1555, 1514, 1470, 1324, 1262, 1235, 1208, 1179, 1094, 1079, 1017, 993, 976, 945, 897, 803, 751, 696, 669, 653 cm⁻¹. MS (DIP), m/e (%): 402 (M⁺, 13), 255 (19), 229 (13), 212 (14), 184 (15), 172 (25), 171 (15), 170 (14), 155 (22), 147 (100), 146 (11), 145 (15), 143 (10), 142 (27), 129 (16), 128 (10), 120 (16), 119 (10), 118 (26), 115 (10), 110 (17), 105 (26), 91 (17), 77 (23), 57 (12). Anal. Calcd for C₂₂H₂₁F₃N₂O₃. 1.0H₂O: C 62.85, H 5.51, N 6.66. Found: C 62.79, H 5.45, N 6.69.

### Example 2: Preparation of 1-(2-oxaadamantan-1-yl)-3-(1-acetylpiperidin-4-yl)urea, I_{f}

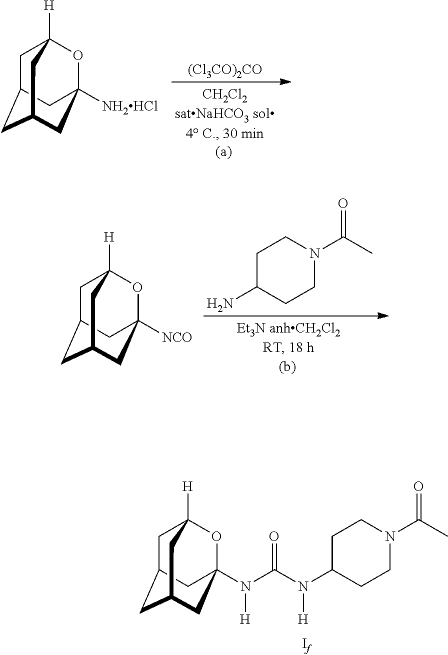

Step (a):

In a three-necked round-bottom flask equipped with a stir bar, low temperature thermometer and gas inlet, triphosgene (392 mg, 1.32 mmol) was added in a single portion to a solution of (2-oxaadamantan-1-yl)amine hydrochloride (500 mg, 2.63 mmol) in DCM (35 mL) and saturated aqueous NaHCO₃ solution (15 mL). The biphasic mixture was stirred vigorously at 4° C. for 30 minutes. Afterwards, the phases were separated and the organic layer was washed with brine (20 mL), dried over anh. Na₂SO₄ and filtered. Evaporation under vacuo provided (2-oxaadamantan-1-yl)isocyanate (408 mg, 86% yield), that was used in next step without further purification. IR (ATR): 2235 (NCO band) cm⁻¹.

Step (b):

Under anhydrous conditions, a solution of (2-oxaadamantan-1-yl)isocyanate (323 mg, 1.80 mmol) in anh. DCM (20 mL) was added to a solution of 1-acetyl-4-aminopiperidine (308 mg, 2.16 mmol) in anh. DCM (10 mL), followed by TEA (0.50 mL, 3.61 mmol). The reaction mixture was stirred at room temperature overnight. The solution was then concentrated under vacuo to give an orange gum (720 mg). Purification by column chromatography (SiO₂, DCM/methanol mixture) gave the title compound I_{f} (300 mg, 52% yield) as a white solid. The analytical sample was obtained by washing with pentane, mp 172-173° C. IR (ATR): 3322, 2920, 2850, 2153, 2000, 1637, 1549, 1428, 1369, 1313, 1264, 1234, 1192, 1139, 1090, 1046, 995, 959, 879, 816, 773, 731 cm¹. MS (DIP), m/e (%): 321 (M⁺, 34), 197 (32), 179 (34), 169 (14), 155 (11), 154 (100), 153 (18), 143 (12), 138 (13), 137 (33), 136 (32), 127 (10), 126 (15), 125 (51), 124 (14), 122 (21), 111 (17), 110 (13), 99 (12), 96 (41), 95 (18), 94 (45), 93 (11), 85 (10), 84 (19), 83 (37), 82 (54), 81 (10), 79 (22), 70 (12), 69 (10), 68 (13), 67 (20), 57 (23), 56 (32), 55 (15). Anal. Calcd for C₁₇H₂₇N₃O₃.0.2H₂O: C 62.82, H 8.50, N 12.93. Found: C 62.70, H 8.59, N 12.74.

### Example 3: Preparation of trans-1-(2-oxaadamantan-1-yl)-3-[4-(4-carboxyphenoxy)cyclohexyl]urea, I_{g}

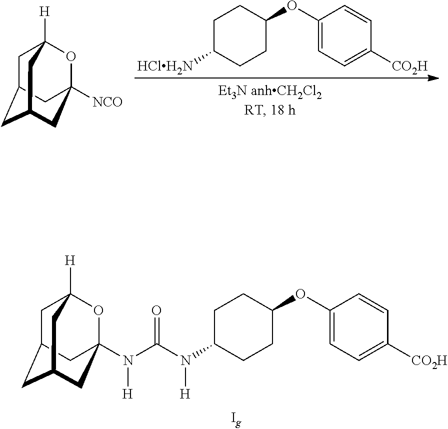

A solution of (2-oxaadamantan-1-yl)isocyanate (400 mg, 2.23 mmol) in anh. DCM (25 mL) was added to a solution of trans-4-(4-aminocyclohexyloxy)benzoic acid hydrochloride (728 mg, 2.68 mmol) in anh. DCM (12 mL), followed by TEA (1.24 mL, 8.94 mmol) under nitrogen. The reaction mixture was stirred at room temperature overnight. Water (50 mL) was then added and the phases were separated. The organic layer was extracted with further water (2×50 mL) and the pH of the combined aqueous phases was adjusted to pH˜2 with 5N HCl solution, prior extraction with DCM (3×50 mL). The combined organic layers were dried over anh. Na₂SO₄, filtered and concentrated under vacuo yielding I_{g} (220 mg, 24% yield) as a white solid. The analytical sample was obtained by crystallization with methanol/diethyl ether, mp 255-275° C. IR (ATR): 3364, 3267, 3198, 3061, 2922, 2559, 2348, 2187, 2068, 2011, 1977, 1672, 1601, 1552, 1443, 1369, 1347, 1320, 1231, 1196, 1172, 1110, 1091, 1049, 1027, 989, 959, 863, 828, 774, 698, 640 cm⁻¹. MS (DIP), m/e (%): 179 (27), 153 (13), 139 (11), 138 (100), 124 (11), 122 (29), 121 (39), 111 (21), 108 (10), 98 (99), 96 (30), 95 (14), 94 (45), 93 (13), 82 (18), 81 (97), 80 (12), 79 (41), 77 (11), 69 (13), 67 (19), 65 (15), 57 (11), 56 (42), 55 (16), 53 (12). Anal. Calcd for C₂₃H₃₀N₂O₅.0.1H₂O: C 66.36, H 7.31, N 6.73. Found: C 66.13, H 7.32, N 6.64.

### Example 4: Preparation of 1-(2-oxaaaaaant-1-yl)-3-(1-benzylpiperidin-4-yl)urea, Iₕ

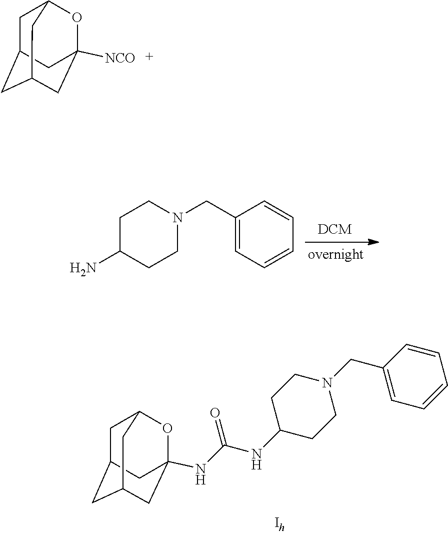

To a solution of 2-oxaadamant-1-yl isocyanate (1.25 g, 6.97 mmol) in DCM (10 mL) was added 1-benzylpiperidin-4-amine (1.60 g, 8.37 mmol). The reaction mixture was stirred at room temperature overnight. The solvents were evaporated under vacuum to give a yellow gum (3.06 g). Column chromatography (dichloromethane/methanol mixtures) gave Iₕ as a yellowish solid (2.54 g, 82% yield). Mp 153-154° C. IR (ATR): 694, 745, 768, 989, 110, 1194, 1225, 1319, 1372, 1441, 1484, 1540, 1664, 1918, 1959, 2918 cm⁻¹. Accurate mass calcd for [C₂₂H₃₁N₃O₂+H]⁺: 370.2489 Found: 370.2488.

### Example 5: Preparation of 1-(1-(4-acetylphenyl)piperidin-4-yl)-3-(2-oxaadamant-1-yl)urea, Iᵢ

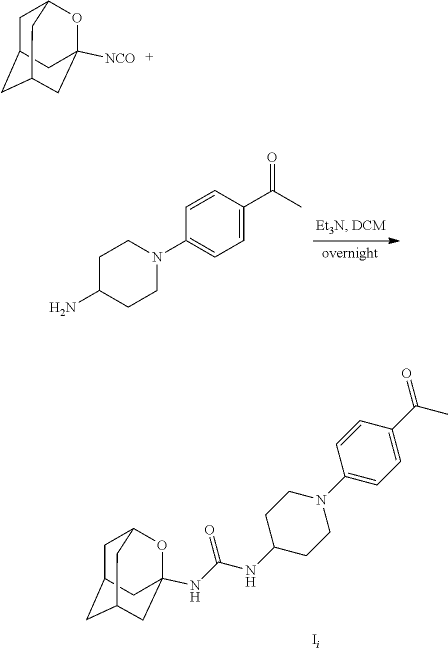

To a solution of 2-oxaadamant-1-yl isocyanate (188 mg, 1.05 mmol) in DCM (5 ml), 1-(4-(4-aminopiperidin-1-yl)phenyl)ethan-1-one (230 mg, 1.05 mmol, prepared following the procedure reported in WO2007016496) and triethylamine (0.15 mL, 1.05 mmol) were added. The reaction mixture was stirred at room temperature overnight. The solvents were evaporated under vacuum to give an orange solid (410 mg). Column chromatography (Dichloromethane/Methanol mixtures) gave Iᵢ as a white solid (183 mg, 45% yield), mp 190-191° C. IR (ATR): 674, 723, 770, 819, 866, 915, 953, 974, 995, 1111, 1134, 1194, 1222, 1279, 1315, 1330, 1475, 1537, 1597, 1653, 1992, 2160, 2341, 2930 cm⁻¹. Anal. Calcd for C₂₃H₃₁N₃O₃. 0.25H₂O: C 68.72%, H 7.90%, N 10.45%. Found: C 68.66%, H 7.78%, N 10.21%.

### Example 6: Preparation of 1-(2-oxaadamantan-1-yl)-3 (benzo[d][1,2,3]thiadiazol-6-yl)urea, Iⱼ

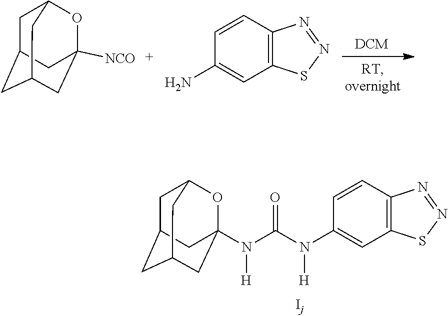

A solution of 2-oxaadamant-1-yl isocyanate (150 mg, 0.84 mmol) in DCM was treated with benzo[d][1,2,3]thiadiazol-6-amine (115 mg, 0.76 mmol). The reaction mixture was stirred at room temperature overnight. The solvents were evaporated under vacuum to give a brownish orange solid (299 mg). Iⱼ was obtained by crystallization from hot EtOAc as a pale orange solid (175 mg, 70% yield), mp 199° C. IR (ATR): 760, 206, 818, 822, 880, 964, 999, 1062, 1088, 1132, 1179, 1194, 1246, 1288, 1320, 1350, 1372, 1405, 1453, 1471, 1537, 1572, 1661, 1681, 1928, 1940, 2069, 2129, 2188, 2263, 2421, 2471, 2560, 2848, 2918, 3111, 3121, 3260, 3338, 3533, 3642, 3776, 3880 cm⁻¹. Anal. Calcd for C₁₆H₁₈N₄O₂S. 0.1 C₄H₈O: C 58.07%, H 5.59%, N 16.52%, S 9.45%. Found: C 58.20%, H 5.46%, N 16.54%, S 9.19%.

### Example 7: Preparation of 1-(2-oxaadamantan-1-yl)-3-(benzo[d]thiazol-2-yl)urea, Iₖ

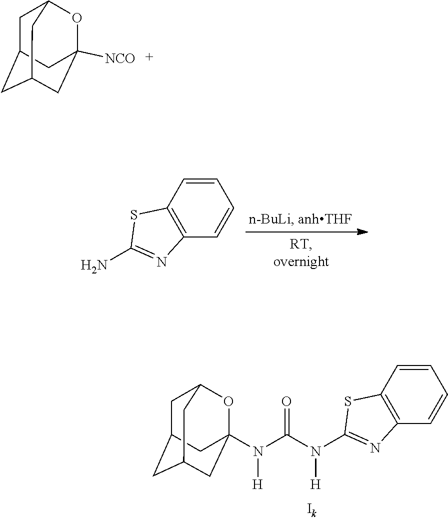

2-amino-1,3-benzothiazole (114 mg, 0.76 mmol) was dissolved in anh. THF (7 mL) under argon and cooled to −78° C. on a dry ice in acetone bath. Then, 2.5 M n-butyllithium in hexanes (0.31 mL, 0.76 mmol) was added dropwise during 20 minutes. Afterwards, the reaction mixture was removed from the dry ice in acetone bath and tempered to 0° C. with an ice bath. Meanwhile, 2-oxaadamant-1-yl isocyanate (150 mg, 0.84 mmol) was dissolved in anh. THF (4 mL) under argon and was continuously added to the reaction mixture. The mixture was stirred at room temperature overnight. Methanol (3 mL) was added to quench any unreacted n-butyllithium. The precipitate formed was filtered and washed with ice-cold THF to afford Iₖ as a white solid (151 mg, 42% yield), mp 240° C. (dec). IR (ATR): 731, 757, 788, 822, 866, 884, 920, 964, 995, 1046, 1093, 1119, 1191, 1248, 1274, 1323, 1341, 1377, 1452, 1514, 1537, 1597, 1718, 1904, 1992, 2036, 2134, 2201, 2852, 2894, 2930, 3064, 3255, 3322 cm. Accurate mass calcd for [C₁₇H₁₉N₃O₂S+H]⁺: 330.1271 Found: 330.1272.

### Example 8: Preparation of 1-(2-oxaadamantan-1-yl)-3-(isoxazol-3-yl)urea, Iₗ

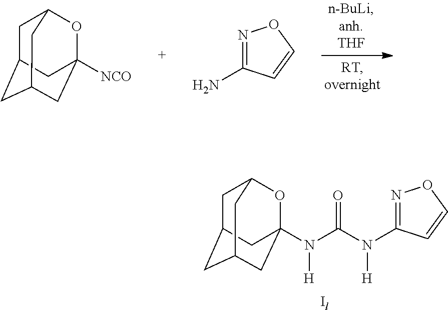

3-aminoisoxazole (103 mg, 1.22 mmol) was dissolved in anh. THF (13 mL) under argon and cooled to −78° C. on a dry ice in acetone bath. Then, 2.5 M n-butyllithium in hexanes (0.50 mL, 1.22 mmol) was added dropwise during 20 minutes. Afterwards, the reaction mixture was removed from the dry ice in acetone bath and tempered to 0° C. with an ice bath. Meanwhile, 2-oxaadamant-1-yl isocyanate (258 mg, 1.34 mmol) was dissolved in anh. THF (6 mL) under argon and was continuously added to the reaction mixture. The mixture was stirred at room temperature overnight. Methanol (4.5 mL) was added to quench any unreacted n-butyllithium. The organic solvents were evaporated under vacuum to give an orange gum (371 mg). Column chromatography (Hexane/Ethyl Acetate mixtures) gave Iₗ as a white solid (90 mg, 22% yield), mp 193° C. IR (ATR): 768, 788, 824, 888, 929, 959, 965, 987, 1014, 1050, 1075, 1093, 1116, 1196, 1260, 1288, 1324, 1377, 1395, 1444, 1475, 1566, 1598, 1672, 1685, 1920, 2005, 2051, 2158, 2215, 2323, 2369, 2851, 2923, 3082, 3179, 3287 cm⁻¹. Anal. Calcd for C₁₃H₁₇N₃O₃: C 59.30%, H 6.51%, N 15.96%. Found: C 59.46%, H 6.70%, N 14.31%.

### Example 9: Preparation of 1-(2-oxaadamantan-1-yl)-3-(1,3,5-triazin-2-yl)urea, Iₘ

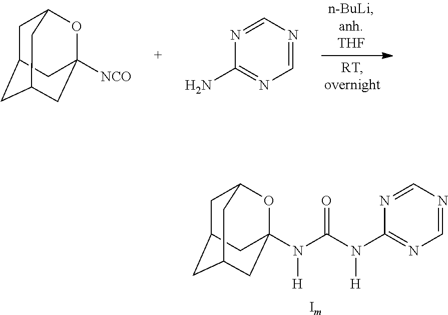

2-amino-1,3,5-triazine (245 mg, 2.55 mmol) was dissolved in anh. THF (20 mL) under argon and cooled to −78° C. on a dry ice in acetone bath. Then, 2.5 M n-butyllithium in hexanes (1.05 mL, 2.55 mmol) was added dropwise during 20 minutes. Afterwards, the reaction mixture was removed from the dry ice in acetone bath and tempered to 0° C. with an ice bath. Meanwhile, 2-oxaadamant-1-yl isocyanate (539 mg, 2.80 mmol) was dissolved in anh. THF (8 mL) under argon and was continuously added to the reaction mixture. The mixture was stirred at room temperature overnight. Methanol (9 mL) was added to quench any unreacted n-butyllithium. A white precipitate formed among the orange solution was filtered and washed with ice-cold THF to afford Iₘ as a white solid (340 mg, 35% yield), mp 157-158° C. IR (ATR): 700, 783, 824, 887, 965, 997, 1080, 1117, 1186, 1194, 1270, 1320, 1343, 1372, 1395, 1402, 1480, 1482, 1502, 1590, 1625, 1700, 2000, 2055, 2170, 2260, 2345, 2546, 2847, 2922, 3233, 3383, 3498 cm⁻¹. Accurate mass calcd for [C₁₃H₁₇N₅O₂+H]⁺: 276.1455. Found: 276.1454.

### Example 10: Preparation of 1-(2-oxaadamant-1-yl)-3-(piperidin-4-yl)urea, Iₙ

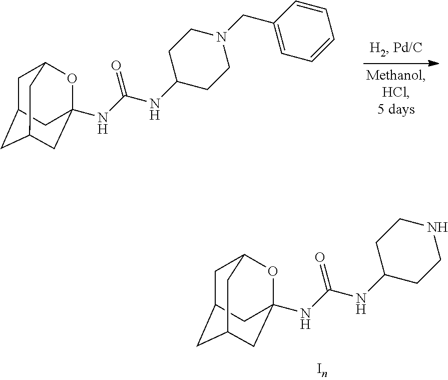 [SMILES: O=C(NC1CCN(Cc2ccccc2)CC1)NC12CC3CC(CC(C3)O1)C2 ; O=C(NC1CCNCC1)NC12CC3CC(CC(C3)O1)C2]

To a solution of 1-(2-oxaadamant-1-yl)-3-(1-benzylpiperidin-4-yl)urea (2.40 g, 6.50 mmol) in methanol (20 rnL), Palladium on carbon 10% wt. (300 mrg) and HCl 37% (1 mL) were added. The reaction mixture was hydrogenated for 5 days. The palladium on carbon was filtered and the solvent was evaporated under vacuum. The crude was dissolved in DCM and washed with 2N NaOH solution (2×30 mL). The organic phase was dried over anh. Na₂SO₄ and filtered. Evaporation under vacuum of the organics gave Iₙ as a white solid (1.28 g, 70% yield). The analytical sample was obtained by crystallization from hot DCM (825 mg), Accurate mass calcd. for [C₁₅H₂₅N₃O₂+H]⁺: 280.2020. Found: 280.2022.

### Example 11: Preparation of 1-(2-oxaadamant-1-yl)-3-(1-(isopropylsulfonyl)piperidin-4-yl)urea, Iₒ

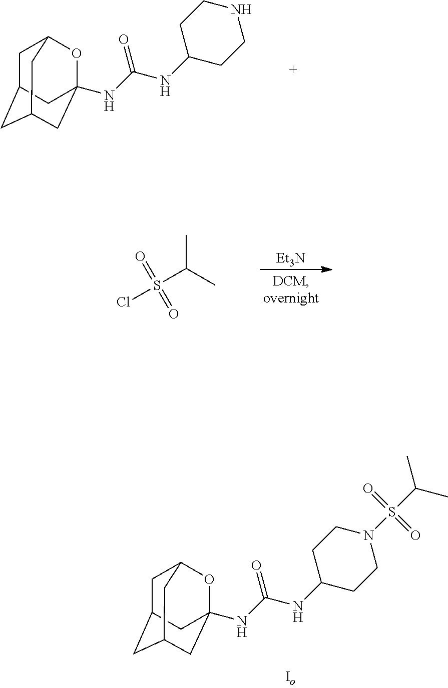

To a solution of 1-(oxaadamant-1-yl)-3-(piperidin-4-yl)urea (250 mg, 0.895 mmol) in DCM (10 mL), triethylamine (0.15 mL, 1.07 mmol) was added. The mixture was cooled down with an ice bath (0° C.) and propane-2-sulfonyl chloride (127 mg, 0.89 mmol) was added dropwise. The reaction mixture was stirred at room temperature overnight and quenched by the addition of HCl solution 37% (2 mL). The organic phase was collected and the aqueous layer was extracted with EtOAc (4×30 mL). The combined organic phases were dried over anh. Na₂SO₄ and filtered. Evaporation of the organics gave an oil that was then dissolved in DCM (20 mL) and washed with 2N NaOH solution (3×20 mL). The organic phase was dried over anh. Na₂SO₄ and filtered. Evaporation under vacuum of the organics gave Iₒ as a white solid (88 mg, 26% yield). The analytical sample was obtained by crystallization from hot DCM as a white solid (60 mg), mp 190-191° C. IR (ATR): 618, 729, 842, 884, 935, 961, 1010, 1041, 1093, 1116, 1132, 1196, 1243, 1269, 1292, 1320, 1374, 1444, 1547, 1635, 2930, 3333 cm⁻¹. Anal. Calcd for C₁₈H₃₁N₃O₄S.0.3 CH₂Cl₂.0.2 CH14: C 54.69%, H 8.01%, N 9.81%. Found: C 54.72%, H 7.91%, N 9.86%.

### Example 12: Preparation of 1-(2-oxaadamant-1-yl)-3-(1-(tetrahydro-2H-pyran-4-carbonyl)piperidin-4-yl)urea, Iₚ

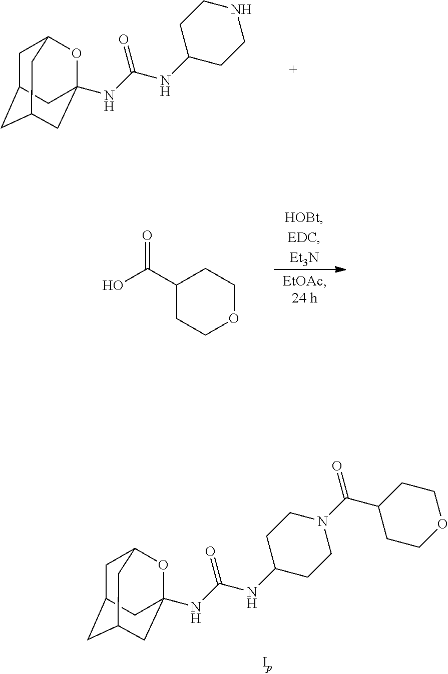 [SMILES: O=C(NC1CCNCC1)NC12CC3CC(CC(C3)O1)C2 ; O=C(O)C1CCOCC1 ; "HOBt,EDC,Et3N" ; O=C(NC1CCN(C(=O)C2CCOCC2)CC1)NC12CC3CC(CC(C3)O1)C2]

To a solution of 1-(2-oxaadamant-1-yl)-3-(piperidin-4-yl)urea (150 mg, 0.53 mmol) in EtOAc (10 mL), tetrahydro-2H-pyran-4-carboxylic acid (70 mg, 0.53 mmol), HOBt (109 mg, 0.80 mmol), EDC (125 mg, 0.80 mmol) and triethylamine (0.15 mL, 1.07 mmol) were added. The reaction mixture was stirred at room temperature for 24 hours. To the resulting suspension was added water (15 mL) and the two phases were separated. The organic phase was washed with saturated aqueous NaHCO₃ solution (15 mL) and brine (15 mL). The combined aqueous phases were extracted with DCM (3×30 mL). The combined organic phases were dried over anh. Na₂SO₄ and filtered. Evaporation under vacuum of the organics gave Iₚ as colorless crystals (190 mg, 90% yield), mp 150-152° C. IR (ATR): 641, 770, 878, 990, 1085, 1121, 1194, 1240, 1318, 1367, 1442, 1550, 1633, 2010, 2067, 2341, 2919 cm⁻¹. Anal. Calcd for C₂₁H₃₃N₃O₄. 0.8 H₂O: C 62.14%, H 8.59%, N 10.35%. Found: C 62.20%, H 8.55%, N 10.38%.

### Example 13: Preparation of 1-(2-oxaadamant-1-yl)-3-(1-(cyclopropanecarbonyl)-piperidin-4-yl)urea, I_{q}

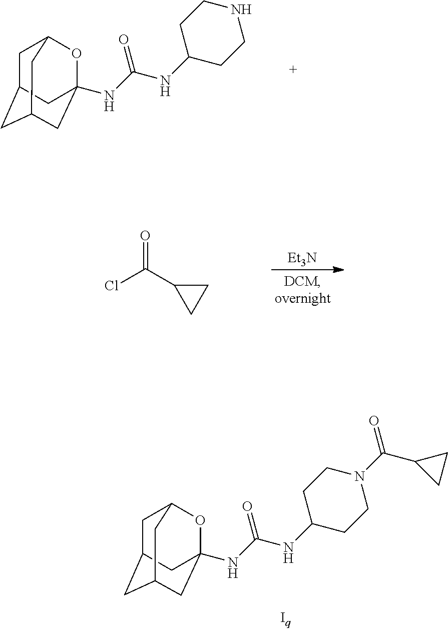 [SMILES: "Et3N" ; O=C(NC1CCNCC1)NC12CC3CC(CC(C3)O1)C2 ; O=C(Cl)C1CC1 ; O=C(NC1CCN(C(=O)C2CC2)CC1)NC12CC3CC(CC(C3)O1)C2]

To a solution of 1-(2-oxaadamant-1-yl)-3-(piperidin-4-yl)urea (300 mg, 1.07 mmol) in DCM (10 mL), cyclopropanecarbonyl chloride (112 mg, 1.07 mmol) and triethylamine (0.18 mL, 1.29 mmol) were added. The reaction mixture was stirred at room temperature overnight and quenched by the addition of aqueous HCl 37% solution (3 mL). The organic phase was collected and the aqueous phase was extracted with EtOAc (4×10 mL). The combined organic phases were washed with NaOH 2N (2×30 mL), dried over anh. Na₂SO₄ and filtered. Evaporation under vacuum of the organics gave I_{q} as a yellow oil (382 mg, 48% yield). The analytical sample was obtained as a white solid (180 mg) by crystallization from hot EtOAc. Mp 197-198° C. IR (ATR): 612, 729, 816, 876, 922, 961, 992, 1085, 1132, 1191, 1219, 1266, 1310, 1369, 1447, 1555, 1604, 1640, 2925, 3307 cm⁻¹. Anal. Calcd for C₁₉H₂₉N₃O₃.0.9H₂O: C 62.75% H 8.54%, N 11.55%. Found: C 63.10%, H 8.57% N 11.15%.

### Example 14: Preparation of 1-(2-oxaadamant-1-yl)-3-(1-nicotinoylpiperidin-4-yl)urea Iᵣ

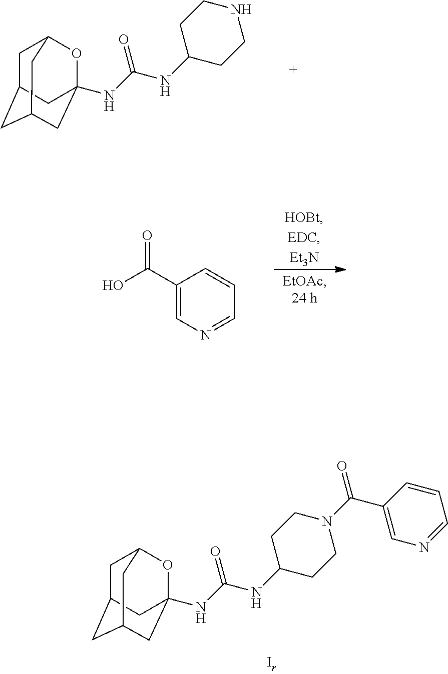 [SMILES: O=C(NC1CCNCC1)NC12CC3CC(CC(C3)O1)C2 ; O=C(O)c1cccnc1 ; "HOBt,EDC,Et3N" ; O=C(NC1CCN(C(=O)c2cccnc2)CC1)NC12CC3CC(CC(C3)O1)C2]

To a solution of 1-(2-oxaadamant-1-yl)-3-(piperidin-4-yl)urea (150 mg. 0.53 mmol) in EtOAc (10 mL), nicotinic acid (66 mg, 0.53 mmol), HOBt (109 mg, 0.805 mmol), EDC (125 mg, 0.80 mmol) and triethylamine (0.15 mL, 1.07 mmol) were added. The reaction mixture was stirred at room temperature for 24 hours. Water (15 mL) was added to the resulting suspension and the two phases were separated. The organic phase was washed with saturated aqueous NaHCO₃ solution (15 mL) and brine (15 mL). The combined aqueous phases were basified with 1N NaOH solution (30 mL) and extracted with DCM (3×30 mL). The combined organic phases were dried over anh. Na₂SO₄ and filtered. Evaporation under vacuum of the organics gave a white solid (140 mg). Column chromatography (Dichloromethane/Methanol mixtures) gave pure Iᵣ as a white solid (63 mg, 32% yield), mp 187-188° C. IR (ATR): 618, 711, 736, 767, 824, 990, 1114, 1132, 1194, 1219, 1245, 1269, 1318, 1367, 1436, 1483, 1537, 1622, 1666, 2051, 2144, 2217, 2919 cm⁻¹. Accurate mass calcd for [C₂₁H₂₈N₄O₃+H]⁺: 385.2234. Found: 385.2238.

### Example 15: Preparation of 1-(2-oxaadamant-1-yl)-3-(1-(2-fluorobenzoyl)piperidin-4-yl)urea, Iₛ

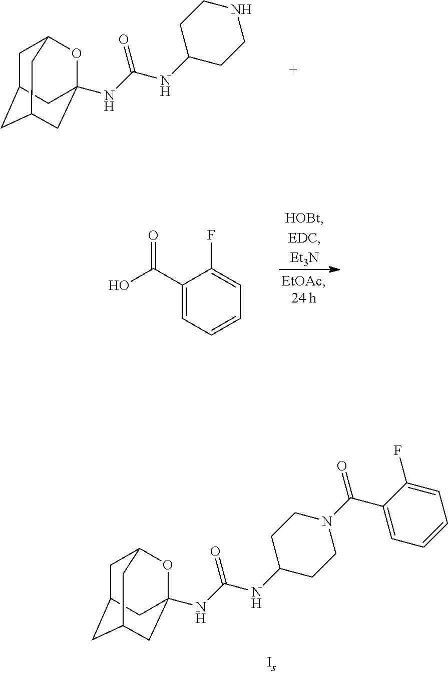 [SMILES: O=C(NC1CCNCC1)NC12CC3CC(CC(C3)O1)C2 ; O=C(O)c1ccccc1F ; "HOBt,EDC,Et3N" ; O=C(NC1CCN(C(=O)c2ccccc2F)CC1)NC12CC3CC(CC(C3)O1)C2]

To a solution of 1-(2-oxaadamant-1-yl)-3-(piperidin-4-yl)urea (120 mg, 0.43 mmol) in EtOAc (10 mL), 2-fluorobenzoic acid (61 mg, 0.43 mmol), HOBt (87 mg, 0.64 mmol), EDC (100 mg, 0.64 mmol) and triethylamine (0.12 mL, 0.86 mmol) were added. The reaction mixture was stirred at room temperature for 24 hours. Water (15 mL) and DCM (20 mL) were added to the resulting suspension and the two phases were separated. The organic phase was washed with saturated aqueous NaHCO₃ solution (15 mL), brine (15 mL), dried over anh. Na₂SO₄ and filtered. Evaporation under vacuum of the organics gave Iₛ as a white solid (131 mg, 77% yield). The analytical sample was obtained as a white solid (111 mg) by crystallization from hot EtOAc. Mp 193-194° C. IR (ATR): 630, 785, 925, 987, 1010, 1093, 1121, 1191, 1243, 1318, 1372, 1447, 1462, 1491, 1555, 1615, 1684, 1974, 2351, 2925, 3338 cm⁻¹. Anal. Calcd for C₂₂H₂₈FN₃O₃: C 65.82%, H 7.03%, N 10.47%. Found: C 65.88%, H 7.25%, N 10.36%.

### Example 16: Preparation of 1-(2-oxaadamant-1-yl)-3-(1-(4-chloro-6-methyl-1,3,5-triazin-2-yl)piperidin-4-yl)urea Iₜ; and 1-(2-oxaadarant-1-yl)-3-(1-(4-methyl-6-(methylamino)-1,3,5-triazin-2-yl)piperidin-4-yl)urea, Iₜₜ

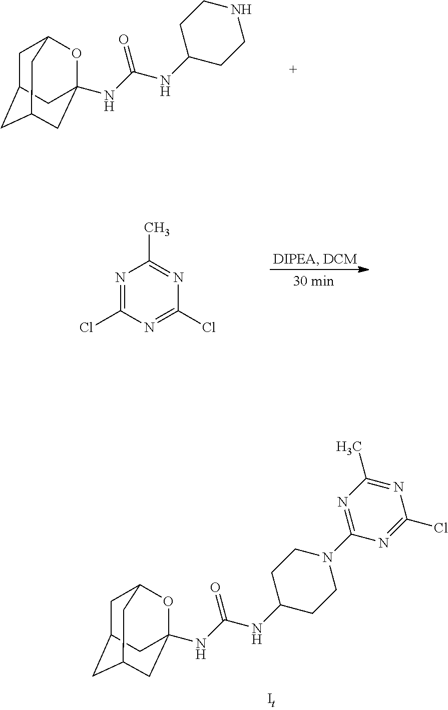

To a solution of 2,4-dichloro-6-methyl-1,3,5-triazine (130 mg, 0.78 mmol) in DCM (4 mL) were added 1-(2-oxaadamantan-1-yl)-3-(piperidin-4-yl)urea (220 mg, 0.78 mmol) and DIPEA (305 mg, 2.36 mmol). The reaction mixture was stirred at room temperature for 30 minutes. The yellow solution was used in the next step without further purification.

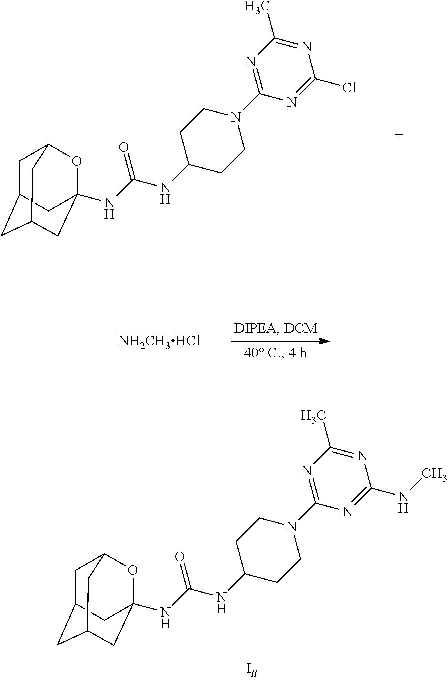 [SMILES: Cc1nc(Cl)nc(N2CCC(NC(=O)NC34CC5CC(CC(C5)O3)C4)CC2)n1 ; "NH2CH3·HCl" ; CNc1nc(C)nc(N2CCC(NC(=O)NC34CC5CC(CC(C5)O3)C4)CC2)n1]

Methylamine hydrochloride (160 mg, 2.36 mmol) and DIPEA (407 mg, 3.15 mmol) were added to the solution of 1-(2-oxaadamantan-1-yl)-3-(1-(4-chloro-6-methyl-1,3,5-triazin-2-yl)piperidin-4-yl)urea in DCM obtained in the previous step. The reaction mixture was stirred at 40′C for 4 hours. The solvent was evaporated under vacuum to give a yellow gum (830 mg). Column chromatography (Dichloromethane/Methanol mixtures) gave Iₜₜ as a white solid (54 mg, 9% yield) and Iₜ as a grey solid (27 mg, 8% yield).

Iₜₜ: Mp 203-204° C. IR (ATR): 653, 803, 880, 993, 1085, 1118, 1189, 1235, 1317, 1366, 1442, 1532, 1644, 1943, 2143, 2337, 2843, 2920 cm⁻¹. Accurate mass calcd for [C₂₀H₃₁N₇O₂+H]⁺: 402.2612. Found: 402.2608.

Iₜ: Mp 196-197° C. IR (ATR): 708, 762, 845, 907, 964, 992, 1075, 1116, 1168, 1194, 1219, 1243, 1271, 1315, 1364, 1444, 1485, 1527, 1578, 1671, 1953, 1974, 1994, 2180, 2335, 2852, 2914 cm⁻¹. Accurate mass calcd for [C₁₉H₂₇ClN₆O₂+H]⁺: 407.1957. Found: 407.1952.

### Example 17: Preparation of 1-(2-oxaadamant-1-yl)-3-(3-chloro-5-trifluoromethoxy)phenyl)urea, Iᵤ

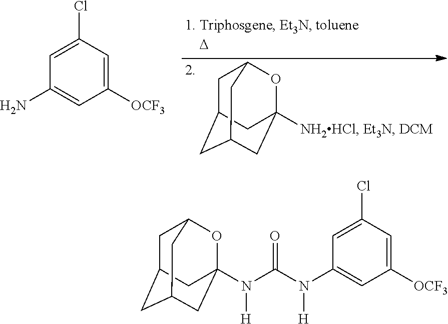

1. A solution of 3-chloro-5-(trifluoromethoxy)aniline (200 mg, 0.94 mmol) in toluene (3 mL) was treated with triphosgene (140 mg, 0.47 mmol). Immediately, triethylamine (0.13 mL, 0.94 mmol) was added and the reaction mixture was stirred at 70° C. for 2 hours. Afterwards, pentane (0.5 mL) was added and a white precipitate was formed. The mixture was filtered and pentane was evaporated under vacuum at room temperature to give the isocyanate in toluene solution that was used in the next step without further purification.

2. To a solution of 3-(trifluoromethoxy)-5-chlorophenyl isocyanate from the previous step were added DCM (5 mL), 2-oxaadamantan-1-amine hydrochloride (161 mg, 0.85 mmol) and triethylamine (0.24 mL, 1.71 mmol). The suspension was stirred at room temperature overnight. The mixture was evaporated under vacuum to give a residue that was then dissolved in DCM (20 mL) and washed with 2N HCl solution. The organic phase was dried over anh. Na₂SO₄ and filtered. Evaporation under vacuum of the organics gave Iᵤ (284 mg, 89% overall yield) as an orange solid. The analytical sample was obtained as a white solid (100 mg) by crystallization from hot DCM, mp 177-178° C. IR (ATR): 672, 747, 935, 964, 995, 1093, 1116, 1152, 1191, 1212, 1248, 1416, 1465, 1550, 1599, 1664, 2930, 3302 cm⁻¹. Anal. Calcd for C₁₇H₁₈ClF₃N₂O₃: C 52.25%, H 4.64%, N 7.17%. Found: C 52.05%, H 4.8%, N 7.02%.

### Example 18: Preparation of 1-(2-oxaadamantan-1-yl)-3-(4-chloro-3-(trifluoromethyl)phenyl)urea, Iᵥ

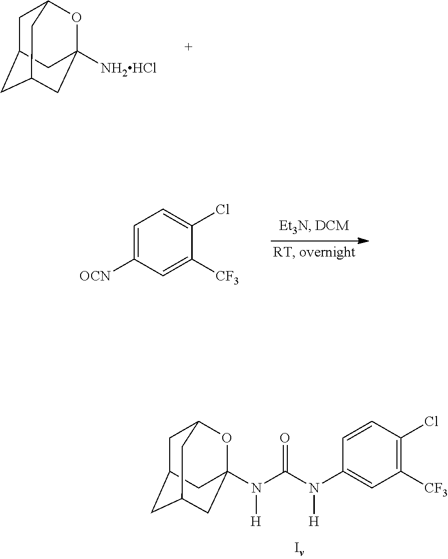

To a solution of the 4-chloro-3-(trifluoromethyl)phenyl isocyanate (191 mg, 0.84 mmol) in DCM were added 2-oxaadamantan-1-amine hydrochloride (145 mg, 0.76 mmol) and triethylamine (0.21 mL, 1.52 mmol). The reaction mixture was stirred at room temperature overnight. The mixture was evaporated under vacuum to give a solid that was then dissolved in EtOAc (20 mL) and washed with 2N HCl solution (10 mL). The organic phase was dried over anh. Na₂SO₄ and filtered. Evaporation under vacuum of the organics gave Iᵥ as a white solid (238 mg, 83% yield). The analytical sample was obtained by crystallization from hot EtOAc (127 mg), mp 196° C. IR (ATR): 661, 721, 765, 785, 824, 835, 881, 930, 961, 987, 1028, 1093, 1114, 1134, 1170, 1191, 1209, 1253, 1289, 1297, 1323, 1374, 1416, 1485, 1550, 1586, 1607, 1671, 2118, 2144, 2217, 2351, 2847, 2925, 3054, 3100, 3235, 3286 cm⁻¹. Anal. Calcd for C₁₇H₁₈ClF₃N₂O₂: C 54.48%, H 4.84%, N 7.47%. Found: C 54.57%, H 4.84%, N 7.64%.

### Example 19: Preparation of 1-(2-oxaadamantan-1-yl)-3-(3-(pentafluoro-λ⁶-sulfanyl)phenyl)urea, Iₓ

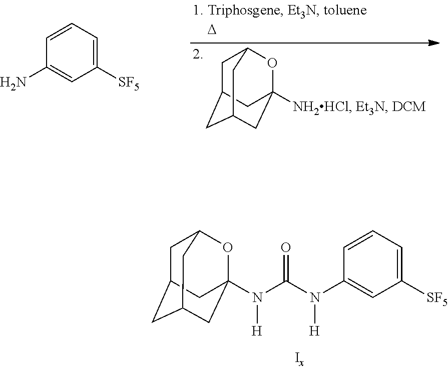

1. A solution of 3-(pentafluoro-λ⁶-sulfanyl)aniline (185 mg, 0.84 mmol) in toluene (3.6 mL) was treated with triphosgene (125 mg, 0.42 mmol). Immediately, triethylamine (0.12 mL, 0.84 mmol) was added and the reaction mixture was stirred at 70° C. for 2 hours. Afterwards, pentane (0.5 mL) was added and a white precipitate was formed. The mixture was filtered and pentane was evaporated under vacuum at room temperature to give the isocyanate in toluene solution that was used in the next step without further purification.

2. To a solution of the 3-(pentafluoro-λ⁶-sulfanyl)phenyl isocyanate were added DCM (5 mL), 2-oxaadamantan-1-amine hydrochloride (145 mg, 0.76 mmol) and triethylamine (0.21 mL, 1.52 mmol). The suspension was stirred at room temperature overnight. The mixture was evaporated under vacuum to give a solid that was then dissolved in DCM (20 mL) and washed with aqueous 2N HCl solution. The organic phase was dried over anh. Na₂SO₄ and filtered. Evaporation under vacuum of the organics gave Iₓ (237 mg, 71% overall yield) as a pale yellow solid. The analytical sample was obtained by crystallization from hot EtOAc as a white solid (75 mg), mp 203° C. IR (ATR): 649, 685, 734, 785, 824, 835, 863, 946, 959, 990, 1093, 1114, 1199, 1250, 1256, 1292, 1318, 1369, 1431, 1478, 1537, 1591, 1671, 1966, 2041, 2930, 3080, 3224, 3286 cm⁻¹. Accurate mass calcd for [C₁₆H₁₉F₅N₂O₂S+H]⁺: 399.1160 Found: 399.1172.

### Example 20: Preparation of methyl 4-(3-(2-oxaadamantan-1-yl)ureido)-2-hydroxybenzoate, I_{y}

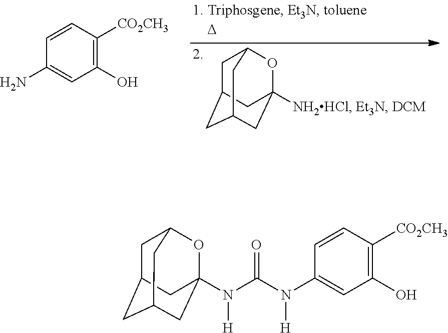

1. A solution of Methyl 4-amino-2-hydroxybenzoate (140 mg, 0.84 mmol) in toluene (3.6 mL) was treated with triphosgene (124 mg, 0.42 mmol). Immediately, triethylamine (0.12 mL, 0.84 mmol) was added and the reaction mixture was stirred at 70° C. for 2 hours. Afterwards, pentane (0.5 mL) was added and a white precipitate was formed. The mixture was filtered and pentane was evaporated under vacuum at room temperature to provide the isocyanate in toluene solution that was used in the next step without further purification.

2. To a solution of the Methyl 2-hydroxy-4-isocyanatobenzoate were added DCM (5 mL), 2-oxaadamantan-1-amine hydrochloride (145 mg, 0.76 mmol) and triethylamine (0.21 mL, 1.52 mmol). The orange solution was stirred at room temperature overnight. The mixture was evaporated under vacuum to give a solid that was then dissolved in DCM (20 mL) and washed with aqueous 2N HCl solution. The organic phase was dried over anh. Na₂SO₄ and filtered. Evaporation under vacuum of the organics gave 240 mg of a yellow solid. Column chromatography (dichloromethane/methanol mixtures) gave I_{y} as a beige solid (47 mg, 16% overall yield), mp 202° C. IR (ATR): 700, 711, 760, 780, 827, 837, 868, 886, 0.928, 956, 992, 1008, 1033, 1095, 1106, 1157, 1194, 1222, 1219, 1253, 1294, 1315, 1333, 1346, 1369, 1405, 1439, 1540, 1599, 1628, 1671, 1976, 2082, 2211, 2273, 2366, 2852, 2925, 3116, 3245 cm⁻¹. Anal. Calcd for C₁₈H₂₂N₂O₅. 0.5H₂O: C 60.83%, H 6.52%, N 7.88%. Found: C 60.87%, H 6.51%, N 7.58%.

### Example 21: Preparation of 1-(2-oxaadamantan-1-yl)-3-(4-chloro-3-(pentafluoro-λ⁶-sulfanyl)phenyl)urea, I_{z}

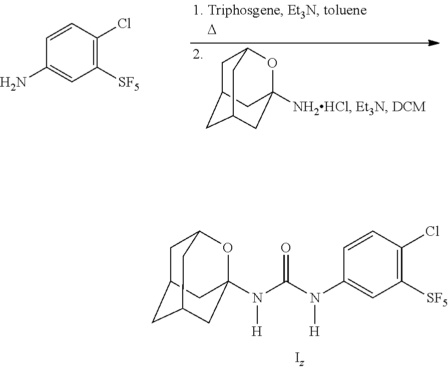

1. A solution of 4-chloro-3-(pentafluoro-λ⁶-sulfanyl)aniline (340 mg, 1.34 mmol) in toluene (4 mL) was treated with triphosgene (199 mg, 0.67 mmol). Immediately, triethylamine (0.82 mL, 1.34 mmol) was added and the reaction mixture was stirred at 70° C. for 2 hours. Afterwards, pentane (1 mL) was added and a white precipitate formed. The mixture was filtered and pentane was evaporated under vacuum at room temperature to give the isocyanate in toluene solution that was used in the next step without further purification.

2. To a solution of the 4-chloro-3-(pentafluoro-λ⁶-sulfanyl)phenyl isocyanate were added DCM (5 mL), 2-oxaadamantan-1-amine hydrochloride (285 mg, 1.50 mmol) and triethylamine (0.38 mL, 2.74 mmol). The suspension was stirred at room temperature overnight. The mixture was evaporated under vacuum to give a solid that was then dissolved in DCM (40 mL) and washed with aqueous 2N HCl solution. The organic phase was dried over anh. Na₂SO₄ and filtered. Evaporation under vacuum of the organics gave 493 mg of a brown solid. Column chromatography (Hexane/Ethyl Acetate mixtures) gave I_{z} as a pale orange solid (116 mg, 20% overall yield), mp 217-218° C. IR (ATR): 646, 672, 700, 742, 757, 783, 814, 827, 853, 899, 935, 964, 995, 1008, 1033, 1067, 1093, 1116, 1132, 1147, 1194, 1250, 1294, 1310, 1354, 1374, 1442, 1480, 1535, 1553, 1589, 1602, 1659, 1958, 1976, 2005, 2015, 2093, 2196, 2852, 2919, 3095, 3317 cm⁻¹. Accurate mass calcd for [C₁₆H₁₈ClF₅N₂O₂S—H]: 431.0625. Found: 431.0629.

### Example 22: In Vitro Determination of sEH Inhibition Activity

The following fluorescent assay was used for determination of the sEH inhibition activity (IC₅₀), with the substrate and comparative control compounds (“standards”) indicated below.

Substrate:

3-(Phenyl-oxiranyl)-acetic acid cyano-(6-methoxy-naphthalen-2-yl)-methyl ester (PHOME; from Cayman Chemical, item number 10009134; CAS 1028430-42-3); cf. N. M. Wolf et al., Anal. Biochem. 2006, vol. 355, pp. 71-80.

Standard 1 (Std 1):

1-(Adamantan-1-yl)-3-(2,3,4-trifluorophenyl)urea. Standard 2 (Std 2): 1-(Adamantan-2-yl)-3-(2,3,4-trifluorophenyl)urea (cf. E. J. North et al., Bioorg. Med. Chem. 2013, vol. 21, pp. 2587-2599).

Solutions:
Assay buffer: Bis/Tris HCl 25 mM pH 7.0 containing 0.1 mg/mL of bovine serum albumin (BSA).
PHOME at 200 μM in DMSO.
Solution of recombinant human sEH (Cayman Chemical, item number 10011669), diluted with assay buffer.
Inhibitors dissolved in DMSO at appropriated concentrations.

Protocol:

In a black 96-well plate (Greiner Bio-One, item number 655900), fill the background wells with 90 μL and the positive control and inhibitor wells with 85 μL of assay buffer. Add 5 μL of DMSO to background and positive control wells, and then add 5 μL of inhibitor solution in inhibitor wells. Add 5 μL of the solution of hsEH to the positive control and inhibitor wells and mix several time. Prepare a 1/21 dilution of the solution of PHOME with assay buffer according to final volume required, and then add 105 μL of each well. Shake carefully the plate for 10 seconds and incubate for 5 minutes at room temperature. Read the appearance of fluorescence with excitation wavelength: 337 nm, and emission wavelength: 460 nm (FLUOStar OPTIMA microplate reader, BMG). The intensity of fluorescence was used to analyze and calculate the IC₅₀ values. Results were obtained by regression analysis from at least three data points in a linear region of the curve. IC₅₀ values are average of minimum three independent replicates. Results are given as means±Standard Error (cf. Table 1).

### Example 23: Determination of Water Solubility

The stock solutions (10⁻² M) of the assayed compounds were diluted to decreased molarity, from 200 μM to 1.02 nM, in 384 well transparent plate (Greiner 781101) with 5% DMSO: 95% PBS buffer. After, they were incubated at 37° C. and solubility S (Table 1) was read after 2 and 4 h in a NEPHELOstar Plus (BMG LABTECH). Results were adjusted to a segmented regression to obtain the maximum concentration in which compounds are soluble.

TABLE 1
sEH inhibition activity, clogP, solubility and melting points of selected
compounds (I) compared with the selected standard

| Compound | IC₅₀ (nM) ± SE | clogP | S (μg/L) | mp (° C.) |
|---|---|---|---|---|
| Std 1 | 7.74 ± 0.06 | 4.04 | 66 | 216-219 |
| Ia | 2.58 ± 0.28 | 2.71 | 77 | 196-198 |
| Ib | 21.3 ± 5.4 | 3.23 | 63 | 195-197 |
| Ic | 8.1 ± 3.2 | 3.76 | 77 | 165-166 |
| Id | 17.5 ± 3.6 | 5.26 | — | 193-195 |
| Ie | 21.3 ± 4.3 | 4.27 | — | 150-152 |
| If | 19.8 ± 6.2 | −0.37 | >100 | 172-173 |
| Ig | 13.4 ± 4.0 | 3.70 | >100 | 255-257 |
| Io | — | 1.06 | >100 | 190-191 |
| Is | — | 1.66 | >100 | 193-194 |
| Iu | — | 4.57 | 45 | 177-178 |
| Iv | — | 4.45 | 60 | 196 |
| Ix | — | 3.65 | >100 | 203 |

### Example 24: Amelioration of the Endoplasmic Reticulum (ER) Stress, Illustrated by the Reduction of Expression of Genes Involved

Cell Culture:

Huh-7 cells were maintained in a humid atmosphere of 5% CO₂ at 37° C. in high glucose (25 mM) Dulbecco's modified Eagle's medium supplemented with 10% heat-inactivated fetal bovine serum, 1% of penicillin/streptomycin (10.000 units/mL of penicillin and 10.000 μg/mL of streptomycin) and 1% of amphotericin B (250 μg/mL).

Cell Treatment:

Huh-7 cells were serum-starved overnight prior treatment. Lipid-containing media were prepared by conjugation of palmitic acid with 2% fatty acid-free BSA, as previously described (cf. L. Salvado et al., “Oleate prevents saturated-fatty-acid-induced ER stress, inflammation, and insulin resistance in skeletal muscle cells through an AMPK-dependent mechanism”, Diabetologia 2013, vol. 56, pp. 1372-1382). For RNA extraction, cells were pre-treated with the inhibitors (final concentration 1 μM) for 1 hour before treatment with palmitate (final concentration 0.5 mM) and inhibitors (final concentration 1 μM). For each condition, at least 3 replicates were performed. Following 48 hours of incubation, RNA were extracted as described below.

Real-Time PCR:

Total RNA in hepatocytes was harvested by TRIsure (Bioline) according to the manufacturer's instructions. The extracted RNA was dissolved in RNase-free water and concentrations of total RNA were quantified using a NanoDrop 2000c spectrophotometer (Thermo Scientific). First-stranded cDNA was synthesized from 0.5 μg total RNA (Life Technologies). Primer Express Software (Applied Biosystems, Foster City, Calif., USA) was used to design the primers examined with SYBR Green I (primers are described in X. Palomer et al., “PPAR3/6 attenuates palmitate-induced endoplasmic reticulum stress and induces autophagic markers in human cardiac cells”, Int. J. Cardiolo. 2014, vol. 174, pp. 110-118). The PCR reaction contained 10 ng of reverse-transcribed RNA, 2× IQ™ SYBRGreen Supermix (BioRad, Barcelona, Spain) and 900 nmol/L concentration of each primer. Optical primer amplification efficiency for each primer set was assessed and a dissociation protocol was carried out to assure a single PCR product. PCR assays were performed on a MiniOpticon™ Real-Time PCR system (BioRad). Thermal cycling conditions were as follows: activation of Taq DNA polymerase at 95° C. for 10 min, followed by 40 cycles of amplification at 95° C. for 15 sec and 60° C. for 1 min. The relative levels of specific mRNA were estimated from the value of the threshold cycle (Ct) of the real-time PCR adjusted by that of a housekeeping gene (GAPDH) through the formula 2ΔΔ^{Ct} (ΔCt=Gene of interest Ct-GAPDH Ct). Cf. Table 2, where CT: control, PAL: palmitate. ***, P<0.001 vs control; ^{#}, P<0.05 vs palmitate; ^{##}, P<0.01 vs palmitate; ^{###}, P<0.001 vs palmitate

TABLE 2
Levels of ATF3, CHOP and BiP mRNA after administration of selected compounds

| Gene | CT | PAL | PAL + Std 2 | PAL + Ia | PAL + Ig |
|---|---|---|---|---|---|
| ATF3 | 100.0 ± 29.4 | 585.5 ± 102.3⁽***⁾ | 225.6 ± 21.2^{(###)} | 191.4 ± 22.6^{(###)} | 286.7 ± 59.9^{(###)} |
| BiP | 100.0 ± 10.7 | 242.9 ± 25.9⁽***⁾ | 135.1 ± 16.2^{(###)} | 129.0 ± 20.5^{(###)} | 174.9 ± 31.4^{(#)} |
| CHOP | 100.0 ± 12.2 | 224.5 ± 9.8⁽***⁾ | 141.8 ± 23.7^{(###)} | 129.9 ± 37.6^{(###)} | 147.6 ± 15.9^{(##)} |

### Example 25: In Vitro Determination of sEH Inhibition Activity in AR42j Cells

The following fluorescent cell-based assay was used for determination of the sEH inhibition activity (IC₅₀), with the Cellular KIT (Cell-Based Assay sEH inhibitor) (Cayman. Ref. 600090).

CBA-Buffer (10×): Cell-Based sEH Assay Buffer 60 mL (Item No. 600091).

CBA Digitonin Solution: 250 μL (Item No. 600092).

CBA sEH Substrate: 100 μL Epoxy-Fluor7 in DMSO (Item No. 600095).

CBA Standard: 100 μL of 100 M CBA 6-methoxy-2-naphtalaldehyde (Item No. 600094).

CBA sEH Positive Control: 10 μL of 1 mg/mL recombinant human sEH (Item No. 600093).

CBA sEH inhibitor: 50 μL of 10 mM AUDA in DMSO (Item No. 600096).

Solutions Preparation:
Assay buffer 1×: add 10 mL of the CBA-Buffer (10×) to 90 mL of distilled water.
Lysis Buffer: add 50 μL of the CBA Digitonin Solution to 10 mL of Buffer Assay 1×.
Substrate Solution: dilute 10 μL of CBA sEH Substrate with 10 mL of Assay Buffer 1×.
Standards: preparation of 7 concentrations of CBA 6-methoxy-2-naphtalaldehyde from 0 until 2 M with Assay Buffer 1×.
sEH Positive Control: Prepare another stock A (1 μg/ml: 1 μL CBA sEH Positive Control+1 mL Assay Buffer 1×). From the already prepared stock A, prepare 250 μL of sEH (10 ng/mL): 2.5 μL sEH stock A+250 μL Assay Buffer 1×).
sEH Inhibitor AUDA: dilute 10 μL CBA sEH Inhibitor with 500 ml Assay Buffer 1×.
Inhibitors dissolved in DMSO at appropriated concentrations.

Protocol:

Seed cells in a 96-well plate at a density of (2×10⁴)-(5×10⁴) cells/well in 100 μL of culture medium with or without compounds to be tested. Incubation of the cells in a CO₂ incubator at 37° C. for 48 hours. Aspirate the culture medium and add 200 μL of Assay Buffer 1× to each well. Centrifuge the plate at 800 rmp for 5 minutes. Aspirate the supernatant and add 100 μL of Lysis Buffer to each well. Incubate with gentle shaking on an orbital shaker for 30 minutes at room temperature. Centrifuge the plate at 3000 rpm for 20 minutes at 4° C. Transfer 90 μL of the supernatants to the 96-Well Solid Plate (black). Add 10 μL Assay Buffer 1× or 10 μL of the AUDA solution to appropriate wells. For positive control wells, add 100 μL of the 10 ng/mL sEH Positive Control to two wells. Add 200 μL of 6-methoxy-2-Naphthaldehyde Standards to corresponding wells of the black plate. Add 100 μL of the sEH Substrate Solution to each well, except the standards. Incubate the plate at 37° C. for 30 minutes. Read the appearance of fluorescence with excitation wavelength: 337 nm, and emission wavelength of each well: 460 nm (Modulus microplate 9300-002, Turner Biosystems). The intensity of fluorescence was used to analyze and calculate inhibition percentages, shown in the Table as average of minimum three independent replicates. Results are given as means±Standard Error (cf. Table 3).

### Example 26: Determination of Cytotoxicity in THLE-2 Cells

Cytotoxic effects of assayed compounds were tested using the immortalized human liver cell line THLE-2 (ATCC CRL-2706). Cells were cultured in BEGM medium (Clonetics # CC-4175) containing all the supplements kit except additional EGF and G418. Medium was completed by adding 0.7 μg/mL phosphoethanolamine, 0.5 ng/mL epidermal growth factor, antibiotics (penicillin and streptomycin) and 10% fetal bovine serum (FBS).

Cells were plated in 96-well black microplates at 10,000 cells/well density and were incubated at 37° C. (5% CO₂, 95% humidity) for 24 h to allow the cells to adhere and form a monolayer. Test compounds were solubilized in 100% DMSO at a concentration curve way and then diluted with cell culture medium containing 10% DMSO. The final concentrations of the test compounds (1% DMSO) ranged from 0-100 μM in a final volume of 200 μL. Microplates were maintained at 37° C. (5% CO₂, 95% humidity) during 3 days. Following this 72 h exposure to test compounds, cell viability in each well was determined by measuring the concentration of cellular adenosine triphosphate (ATP) using the ATP1Step Kit as described by the manufacturer (Perkin-Elmer). In a typical procedure, 50 μL of cell reagent is added to all wells of each test plate followed by incubation for 10 min at room temperature on an orbital shaker. ATP concentration was determined by reading chemical luminescence using the Envision plate reader (PerkinElmer). The percentage of viable cells relative to the non-drug treated controls was determined for each well and LC₅₀ values were calculated as concentrations projected to kill 50% of the cells following a 72 h exposure, an average of minimum two independent replicates. Results are given as means±Standard Error (cf. Table 3).

### Example 27: Parallel Artificial Membrane Permeation Assays—Blood-Brain Barrier

To evaluate the brain penetration of the different compounds, a parallel artificial membrane permeation assay for blood-brain barrier (PAMPA-BBB) was used, following the method described by L. Di et al., “High throughput artificial membrane permeability assay for blood-brain barrier”, Eur. J. Med. Chem. 2003, vol. 38. pp. 223-232. The in vitro permeability (Pₑ) of the test compounds through lipid extract of porcine brain membrane was determined. Assayed compounds were tested using a mixture of PBS:EtOH (70:30). Assay validation was made by comparing the experimental permeability with the reported values of the commercial drugs by bibliography and lineal correlation between experimental and reported permeability of the fourteen commercial drugs using the parallel artificial membrane permeation assay was evaluated (y=1.537 x−0.967; R²=0.9382). From this equation and taking into account the limits established by Di et al. for BBB permeation, the ranges of permeability were established, as follows. Compounds of high BBB permeation (CNS+): Pe (10⁶ cm s⁻¹)>5.181; compounds of low BBB permeation (CNS−): Pe (10⁶ cm s⁻¹)<2.107; and compounds of uncertain BBB permeation (CNS+/−): 5.181>Pe (10⁻⁶ cm s⁻¹)>2.107. The permeability results from the assayed compounds are averages of three independent replicates and a predictive penetration in the CNS is also given. Qualitative results are shown in Table 3 (n.d.=not determined).

TABLE 3
sEH inhibition activity in cell culture, cytotoxicity, and CNS prediction

| Compound | % Inhn. ± SE (100 μM) | LC₅₀ (μM) | CNS prediction |
|---|---|---|---|
| Std 1 | 56.6 ± 11.0 | n.d. | — |
| Ia | 35.8 ± 4.7 | >100 | CNS+ |
| Ib | n.d. | >100 | CNS+ |
| Ic | n.d. | >100 | CNS+ |
| Ig | 58.3 ± 3.2 | >100 | CNS+ |
| If | 42.1 ± 5.0 | >100 | n.d. |
| Io | 40.89 ± 1.99 | >100 | CNS− |
| Is | 45.3 ± 3.5 | >100 | CNS− |
| Iv | 51.6 ± 6.6 | 45.8 ± 3.8 | CNS+ |

1. A compound of formula I 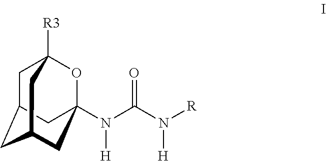
or a stereoisomer or a pharmaceutically acceptable salt thereof, wherein:
R3 is a radical selected from the group consisting of H, C₁-C₃ alkyl, cyclohexyl and phenyl;
R is a radical —[CH₂]ₙ—Y, wherein n is an integer between 0 and 15, and in the —[CH₂]ₙ— biradical an integer between 0 and n/3 of the methylene groups are optionally replaced by oxygen atoms in such a way that there are not two oxygen atoms which are adjacent;
Y is a radical selected from the group consisting of: phenyl; a substituted phenyl; cyclohexyl; a substituted cyclohexyl; a piperidinyl; a substituted piperidinyl; a C- or N radical from a 5- or 6-membered aromatic heterocycle; and a C- or N-radical from a 5- or 6-membered aromatic heterocycle fused with a benzene ring;
with the proviso that I is not 1-(2-oxaadamantan-1-yl)-3-(3,4-dichlorophenyl)urea.

2. The compound according to claim 1, wherein Y is a radical selected from the group consisting of:
di- and tri-substituted phenyl radicals, wherein the two or three substituents, equal or different, are independently selected from the group consisting of F, Cl, SF₅, CF₃, OH, OCF₃, C₁-C₃ alkyl, and (C₁-C₃)—OCO;
a C- or N-radical from a 5- or 6-membered aromatic heterocycle, having in the cycle one, two or three atoms of N, S or O;
a C- or N-radical from a 5- or 6-membered aromatic heterocycle having in the cycle one, two or three atoms of N, S or O, which is fused with a benzene ring; and
radicals having one of the following four general formulas, wherein bonds crossing positions 3 and 4 of the phenyl and cyclohexyl rings mean substitution either in position 3 or in position 4 of the radical ring; 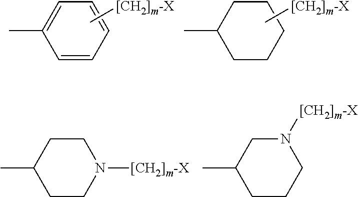
wherein m is an integer between 0 and 15, and in the —[CH₂]ₘ— biradical an integer between 0 and m/3 methylene groups are optionally replaced by oxygen atoms in such a way that there are not two oxygen atoms which are adjacent; and
X is a radical selected from the group consisting of:
H, F, Cl, SF₅, CF₃, OCF₃, OH, CN, COOH, C₁-C₃ alkyl, (C₁-C₃ alkyl)CO, (C₁-C₃ alkyl)SO₂;
phenyl, benzoyl, phenoxy, mono-substituted phenyl, mono-substituted benzoyl and mono-substituted phenoxy wherein the substituent is selected from the group consisting of F, Cl, CHO, COCH₃, COOH, and H₂NSO₂;
(C₁-C₁₅ linear alkyl)O, (C₄-C₁₅ linear alkyl)CO, (C₁-C₁₅ linear alkyl)OCO, (C₁-C₁₅ linear alkyl)NHCO, (C₁-C₁₅ linear alkyl)CONH, (C₄-C₁₅ linear alkyl)SO₂, (C₁-C₁₅ linear alkyl)NHSO₂, (C₁-C₁₅ linear alkyl)SO₂NH;
(C₃-C₆ carbocyclyl)O, (C₃-C₆ carbocyclyl)CO, (C₃-C₆ carbocyclyl)OCO, (C₃-C₆ carbocyclyl)NHCO, (C₃-C₆ carbocyclyl)CONH, (C₃-C₆ carbocyclyl)SO₂, (C₃-C₆ carbocyclyl)NHSO₂, (C₃-C₆ carbocyclyl)SO₂NH;
(5/6-membered-N/O-heterocyclyl)O, (5/6-membered-N/O-heterocyclyl)CO, (5/6-membered-N/O-heterocyclyl)OCO, (5/6-membered-N/O—)NHCO, (5/6-membered-N/O-heterocyclyl)CONH, (5/6-membered-N/O-heterocyclyl) SO₂, (5/6-membered-N/O-heterocyclyl)NHSO₂, and (5/6-membered-N/O-heterocyclyl)SO₂NH; wherein 5/6-membered-N/O-heterocyclyl is a C- or N-radical from a 5- or 6-membered heterocycle, the heterocycle being aromatic or non-aromatic, the heterocycle having in the cycle one, two or three atoms of N, S or O; wherein the 5/6-membered-N/O-heterocyclyl radical is optionally substituted by one or two substituents, equal or different, independently selected from the group consisting of F, Cl, CF₃, C₁-C₃ alkyl, and (C₁-C₃ alkyl)NH.

3. The compound according to claim 2, wherein Y is a radical selected from the group consisting of:
di- and a tri-fluorosubstituted phenyl radicals;
4-chloro-3-trifluoromethylphenyl;
3-chloro-4-trifluoromethylphenyl;
4-fluoro-3-trifluoromethylphenyl;
3-fluoro-4-trifluoromethylphenyl; and
radicals having the four general formulas as defined in claim 2, wherein X is a radical selected from the group consisting of:
H, F, Cl, CF₃, OCF₃, OH, CN, COOH;
phenyl, phenoxy, mono-substituted phenyl and mono-substituted phenoxy, wherein the substituent is COOH, Cl or H₂NSO₂;
(C₁-C₁₅ linear alkyl)O, (C₁-C₁₅ linear alkyl)CO, (C₁-C₁₅ linear alkyl)OCO,
(C₁-C₁₅ linear alkyl)NHCO, (C₁-C₁₅ linear alkyl)CONH, (C₁-C₁₅ linear alkyl)SO₂ (C₁-C₁₅ linear alkyl)NHSO₂, (C₁-C₁₅ linear alkyl)SO₂NH;
(5/6-membered-N/O-heterocyclyl)O, (5/6-membered-N/O-heterocyclyl)CO, (5/6-membered-N/O-heterocyclyl)OCO, (5/6-membered-N/O-heterocyclyl)-NHCO, (5/6-membered-N/O-heterocyclyl)CONH;
(5/6-membered-N/O-heterocyclyl)SO₂, (5/6-membered-N/O-heterocyclyl)NHSO₂, and (5/6-membered-N/O-heterocyclyl)SO₂NH; wherein 5/6-membered-N/O-heterocyclyl is a C-radical or a N-radical from any 5- or 6-membered heterocycle, the heterocycle being aromatic or non-aromatic, and the heterocycle having in the cycle either one N atom, or two N atoms, or simultaneously one N atom and one O atom.

4. The compound according to claim 1, wherein in radical R integer n is between 0 and 3, and consequently only one methylene group is optionally replaced by an oxygen atom.

5. The compound according to claim 1, wherein in radical R integer n is 0, and consequently R═Y.

6. The compound according to claim 2, wherein Y is a radical having the following formula:

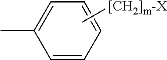

7. The compound according to claim 2, wherein Y is a radical having the following formula:

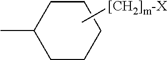

8. The compound according to claim 2, wherein Y is a radical having the following formula:

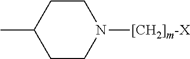

9. The compound according to claim 2, wherein integer m is between 0 and 3, and consequently only one methylene group is optionally replaced by an oxygen atom.

10. The compound according to claim 2, wherein integer m is 0.

11. The compound according to claim 2, wherein X is a radical selected from the group consisting of: H, F, Cl, CF₃, OCF₃, OH, CN, COOH, (C₁-C₅ linear alkyl)O, (C₁-C₅ linear alkyl)CO, (C₁-C₅ linear alkyl)OCO, (C₁-C₅ linear alkyl)NHCO, (C₁-C₅ linear alkyl)CONH, (C₁-C₅ linear alkyl)SO₂, (C₁-C₅ linear alkyl)NHSO₂, (C₁-C₅ linear alkyl)SO₂NH, 2-pyridinyl, 3-pyridynyl, 4-pyridynyl, 4-morpholinyl, phenyl, phenoxy, a mono-substituted phenyl and a mono-substituted phenoxy, whose substitution in the two latter cases is done by a radical selected from the group consisting of COOH, Cl and H₂NSO₂.

12. The compound according to claim 1, wherein Y is a radical selected from the group consisting of:
a tri-fluorosubstituted phenyl radical;
4-chloro-3-trifluoromethylphenyl;
3-chloro-4-trifluoromethylphenyl;
4-fluoro-3-trifluoromethylphenyl, and
3-fluoro-4-trifluoromethylphenyl.

13. The compound according to claim 1, wherein R3 is H.

14. The compound according to claim 1, which is selected from the group consisting of:
1-(2-oxaadamantan-1-yl)-3-(2,3,4-trifluorophenyl)urea;
1-(3-methyl-2-oxaadamantan-1-yl)-3-(2,3,4-trifluorophenyl)urea;
1-(3-ethyl-2-oxaadamantan-1-yl)-3-(2,3,4-trifluorophenyl)urea;
1-(3-cyclohexyl-2-oxaadamantan-1-yl)-3-(2,3,4-trifluorophenyl)urea;
1-(3-phenyl-2-oxaadamantan-1-yl)-3-(2,3,4-trifluorophenyl)urea;
1-(2-oxaadamantan-1-yl)-3-(1-acetylpiperidin-4-yl)urea; and
trans-1-(2-oxaadamantan-l-yl)-3-[4-(4-carboxyphenoxy)cyclohexyl]urea.

15. A pharmaceutical composition comprising a therapeutically effective amount of a compound as defined in claim 1, or a stereoisomer or a pharmaceutically acceptable salt thereof, and adequate amounts of pharmaceutically acceptable excipients.

16.-18. (canceled)

19. A method of treatment of an animal-including a human-suffering from a soluble epoxide hydrolase mediated disease, comprising the administration of a therapeutically effective amount of a compound as defined in claim 1, or a stereoisomer or a pharmaceutically acceptable salt thereof, together with adequate amounts of pharmaceutically acceptable excipients.

20. The method according to claim 19, wherein the soluble epoxide hydrolase mediated disease is selected from the group consisting of hypertension, atherosclerosis, pulmonary diseases, kidney diseases, stroke, pain, neuropathic pain, inflammation, pancreatitis, immunological disorders, eye diseases, cancer, obesity, diabetes, metabolic syndrome, preeclampsia, anorexia nervosa, depression, erectile dysfunction, wound healing, NSAID-induced ulcers, emphysema, scrapie and Parkinson's disease.

21. The compound according to claim 6, wherein:
R3 is H;
m is 0; and
X is a radical selected from the group consisting of: H, F, Cl, CF₃, OCF₃, OH, CN, COOH, (C₁-C₅ linear alkyl)O, (C₁-C₅ linear alkyl)CO, (C₁-C₅ linear alkyl)OCO, (C₁-C₅ linear alkyl)NHCO, (C₁-C₅ linear alkyl)CONH, (C₁-C₅ linear alkyl)SO₂, (C₁-C₅ linear alkyl)NHSO₂, (C₁-C₅ linear alkyl)SO₂NH, 2-pyridinyl, 3-pyridynyl, 4-pyridynyl, 4-morpholinyl, phenyl, phenoxy, a mono-substituted phenyl and a mono-substituted phenoxy, whose substitution in the two latter cases is done by a radical selected from the group consisting of COOH, Cl and H₂NSO₂.

22. The compound according to claim 7, wherein:
R3 is H;
m is 0; and
X is a radical selected from the group consisting of: H, F, Cl, CF₃, OCF₃, OH, CN, COOH, (C₁-C₅ linear alkyl)O, (C₁-C₅ linear alkyl)CO, (C₁-C₅ linear alkyl)OCO, (C₁-C₅ linear alkyl)NHCO, (C₁-C₅ linear alkyl)CONH, (C₁-C₅ linear alkyl)SO₂, (C₁-C₅ linear alkyl)NHSO₂, (C₁-C₅ linear alkyl)SO₂NH, 2-pyridinyl, 3-pyridynyl, 4-pyridynyl, 4-morpholinyl, phenyl, phenoxy, a mono-substituted phenyl and a mono-substituted phenoxy, whose substitution in the two latter cases is done by a radical selected from the group consisting of COOH, Cl and H₂NSO₂.

23. The compound according to claim 8, wherein:
R3 is H;
m is 0; and
X is a radical selected from the group consisting of: H, F, Cl, CF₃, OCF₃, OH, CN, COOH, (C₁-C₅ linear alkyl)O, (C₁-C₅ linear alkyl)CO, (C₁-C₅ linear alkyl)OCO, (C₁-C₅ linear alkyl)NHCO, (C₁-C₅ linear alkyl)CONH, (C₁-C₅ linear alkyl)SO₂, (C₁-C₅ linear alkyl)NHSO₂, (C₁-C₅ linear alkyl)SO₂NH, 2-pyridinyl, 3-pyridynyl, 4-pyridynyl, 4-morpholinyl, phenyl, phenoxy, a mono-substituted phenyl and a mono-substituted phenoxy, whose substitution in the two latter cases is done by a radical selected from the group consisting of COOH, Cl and H₂NSO₂.
